(19) United States
(12) Patent Application Publication
(10) Pub. No.: US 2026/0265765 A1
(43) Pub. Date: Sep. 10, 2026

(54) GENE EXPRESSION METHOD

(71) Applicants: KABUSHIKI KAISHA TOYOTA CHUO KENKYUSHO, Nagakute-shi, Aichi-ken (JP); TOYOTA JIDOSHA KABUSHIKI KAISHA, Toyota-shi, Aichi-ken (JP)

(72) Inventors: Noriko YASUTANI, Nagoya-shi (JP); Toru ONISHI, Toyota-shi (JP)

(73) Assignees: KABUSHIKI KAISHA TOYOTA CHUO KENKYUSHO, Nagakute-shi, Aichi-ken (JP); TOYOTA JIDOSHA KABUSHIKI KAISHA, Toyota-shi, Aichi-ken (JP)

(21) Appl. No.: 19/167,220

(22) PCT Filed: Oct. 26, 2023
(86) PCT No.: PCT/JP2023/038726

§ 371 Date: Sep. 19, 2025

(30) Foreign Application Priority Data
Mar. 27, 2023 (JP) 2023-050539

(51) Int. Cl. C12N 15/81; C12N 9/16; C12N 15/70; C12R 1/19; C12R 1/865

(52) U.S. Cl. CPC C12N 15/81; C12N 9/16; C12N 15/70; C12N 2510/00; C12R 2001/19; C12R 2001/865

(57) ABSTRACT

A gene expression method includes: providing a target gene-introducing plasmid where a target gene that is to be introduced into and to be expressed in a host cell and an endonuclease gene that specifically recognizes a specific sequence are placed to be expressible under control of one or more inducible promoters configured to induce simultaneous expression and where one or more specific sequences are introduced; transforming the host cell with the target gene-introducing plasmid; and culturing the host cell under an inducing condition of the inducible promoter, so as to cause expression of the target gene and the endonuclease gene and to cause an expressed endonuclease to cut the specific sequence and thereby remove the target gene-introducing plasmid.

### CROSS REFERENCE TO RELATED APPLICATIONS

This is a National Phase Application based on PCT Application PCT/JP2023/038726 filed on Mar. 27, 2023. The disclosure of the prior application is hereby incorporated by reference herein in its entirety.

### SEQUENCE LISTING

The present application contains a Sequence Listing that has been submitted electronically and is hereby incorporated by reference in its entirety. The electronic Sequence Listing is named Sequence_Listing_ST26, was created on Sep. 19, 2025, and is 72,851 bytes in size.

### TECHNICAL FIELD

The present disclosure relates to a gene expression method.

### BACKGROUND ART

There have been a variety of conventionally known techniques with regard to introduction of a gene into a host cell. For example, Patent Literature 1 discloses a technique that incorporates a linear genome-introducing nucleic acid fragment into a plasmid for assisting transformation by homologous recombination, introduces this plasmid into a host, subsequently incises a gene included in the linear genome-introducing nucleic acid fragment from the plasmid for assisting transformation by the action of an endonuclease within a host cell, and incorporates the incised gene into a host genome by homologous recombination. Using the plasmid for assisting transformation as described above is allowed to enhance the efficiency of transformation of the linear genome-introducing nucleic acid fragment. Furthermore, the incorporation of the incised gene into the host genome enables a desired gene to be stably introduced into the host cell.

### CITATION LIST

### Patent Literatures

Patent Literature 1: Japanese Patent 2021-000041A

### SUMMARY

### Technical Problem

In the procedure described in the above Patent Literature 1, an available method of enhancing the efficiency of incorporating a desired gene into the host genome by homologous recombination is, for example, a method that further introduces a plasmid including a gene of an enzyme, which accelerates a homologous recombination reaction (recombinase gene), into a host cell and causes the recombinase gene to be expressed in the host cell. In this case, there is a need to express the recombinase gene and activate a recombinase at a desired timing for performing the homologous recombination to incorporate the desired gene into the host genome. Furthermore, there is a possibility that the continuous expression of the recombinase gene even after conclusion of the homologous recombination of the desired gene causes an undesired recombination reaction to proceed within the host cell. With a view to reducing such a disadvantage, there is a need to make the expression of the recombinase gene off after conclusion of the homologous recombination of the desired gene. For example, removal of the plasmid including the recombinase gene from the host cell certainly makes the expression of the recombinase gene off. A troublesome operation is, however, required for the removal of the plasmid, and in some cases, there is a difficulty in employing such a troublesome operation. There is accordingly a requirement for a simpler operation to introduce a recombinase gene into a host cell and express the gene at a desired timing and to remove the introduced recombinase gene from the host cell when the recombinase gene becomes unnecessary. Such a problem is not limited to the process of causing the recombinase gene to be introduced into and to be expressed in the host cell but is commonly found in the process of causing a certain target gene to be introduced into a host cell and to be transiently expressed at a desired timing.

### Solution to Problem

The present disclosure may be implemented by the following aspects:
(1) According to one aspect of the present disclosure, there is provided a gene expression method. This gene expression method comprises providing a target gene-introducing plasmid where a target gene that is to be introduced into and to be expressed in a host cell and an endonuclease gene that specifically recognizes a specific sequence are placed to be expressible under control of one or more inducible promoters configured to induce simultaneous expression and where one or more specific sequences are introduced; transforming the host cell with the target gene-introducing plasmid; and culturing the transformed host cell under an inducing condition of the inducible promoter, so as to cause expression of the target gene and the endonuclease gene and to cause an endonuclease expressed by the expression of the endonuclease gene to cut the specific sequence and thereby remove the target gene-introducing plasmid.

The gene expression method of this aspect enables the target gene to be expressed at a desired timing by a simple operation of introducing the target gene-introducing plasmid into the host cell and inducing the inducible promoter and subsequently enables the target gene introducing plasmid to be removed without requiring any further operation. In other words, the gene expression method of this aspect causes the target gene introducing plasmid to be automatically removed after expression of the target gene and thereby makes the expression of the target gene off.
(2) In the gene expression method of the above aspect, a gene for modifying a nucleic acid included in the host cell may be used as the target gene. The configuration of this aspect suppresses the target gene from being continuously expressed after elapse of the desired timing and thereby suppresses an undesired reaction from proceeding within the host cell by the longer expression of the target gene, which is the gene for modifying the nucleic acid.
(3) In the gene expression method of the above aspect, a gene of an enzyme that modifies a genomic DNA of the host cell by a homologous recombination reaction may be used as the target gene. The configuration of this aspect suppresses the target gene from being continuously expressed after elapse of the desired timing and thereby suppresses an undesired reaction from proceeding within the host cell by the longer expression of the target gene, which is the gene of the enzyme that modifies the genomic DNA of the host cell by the homologous recombination reaction.
(4) In the gene expression method of the above aspect, a gene coding a λ-Red recombinase system may be used as the target gene. The configuration of this aspect suppresses the target gene from being continuously expressed after elapse of the desired timing and thereby suppresses an undesired reaction from proceeding within the host cell by the longer action of the A-Red recombinase system.
(5) In the gene expression method of the above aspect, a plasmid that has two or more specific sequences and that includes a sequence, which is introduced in between the two specific sequences and which allows for homologous recombination with the genomic DNA of the host cell, may be used as the target gene-introducing plasmid. The configuration of this aspect causes the target gene and the endonuclease gene to be expressed by introduction of the target gene-introducing plasmid into the host cell and induction of the inducible promoter and then causes the expressed endonuclease to cut the specific sequence, so as to excise the sequence that allows for homologous recombination with the genomic DNA of the host cell, from the target gene-introducing plasmid. This configuration does not require any additional operation for separately providing a vector that includes a sequence allowing for homologous recombination with the genomic DNA of the host cell and for introducing the vector into the host cell and thereby simplifies the operation for introducing a nucleic acid sequence by the homologous recombination with the genomic DNA of the host cell.
(6) In the gene expression method of the above aspect, a homing endonuclease gene may be used as the endonuclease gene. The configuration of this aspect allows a specific sequence selected corresponding to a homing endonuclease to be provided in the target gene-introducing plasmid and thereby facilitates an operation of removing the target gene-introducing plasmid after the expression and the action of the target gene.
(7) In the gene expression method of the above aspect, a single inducible promoter may be used as the one or more inducible promoters configured to induce simultaneous expression. The configuration of this aspect furthermore simplifies the configuration of the target gene-introducing plasmid and enables the expression of the target gene and the expression of the endonuclease gene to be induced simultaneously.
(8) In the gene expression method of the above aspect, a prokaryotic cell may be used as the host cell, and a plasmid where the target gene and the endonuclease gene are placed in series downstream of the single inducible promoter, may be used as the target gene-introducing plasmid. The configuration of this aspect causes the inducible promoter, the target gene, and the endonuclease gene to function as an operon and enables both the target gene and the endonuclease gene to be transcribed to mRNA and to be expressed at the same timing by induction of the inducible promoter.
(9) In the gene expression method of the above aspect, a eukaryotic cell may be used as the host cell, a bidirectional promoter may be used as the single inducible promoter, and a plasmid where the target gene and the endonuclease gene are placed across the bidirectional promoter to be located on respective sides of the bidirectional promoter and are connected such as to be simultaneously expressible by induction of the bidirectional promoter, may be used as the target gene-introducing plasmid. The configuration of this aspect enables the target gene and the endonuclease gene to be transcribed and expressed at the same timing by induction of the inducible promoter.
(10) In the gene expression method of the above aspect, a eukaryotic cell may be used as the host cell, and a plasmid where the target gene and the endonuclease gene are placed in series downstream of the single inducible promoter and are connected with each other by a linker such as to be controlled by the single inducible promoter and to be transcribed simultaneously, may be used as the target gene-introducing plasmid. The configuration of this aspect enables the target gene and the endonuclease gene to be transcribed and expressed at the same timing by induction of the inducible promoter.

The present disclosure may be implemented by a variety of aspects other than those described above: for example, a plasmid used to perform the gene expression method and a method of removing a gene that is transiently expressed.

### BRIEF DESCRIPTION OF DRAWINGS

FIG. 1 is an explanatory diagram illustrating a gene expression method according to a first embodiment;

FIG. 2 is an explanatory diagram illustrating the schematic configuration of a target gene-introducing plasmid;

FIG. 3 is an explanatory diagram illustrating one example of a conventionally known gene expression method;

FIG. 4 is an explanatory diagram illustrating the schematic configuration of a target gene-introducing plasmid;

FIG. 5 is an explanatory diagram illustrating a gene expression method according to a third embodiment;

FIG. 6 is an explanatory diagram illustrating the schematic configuration of a target gene-introducing plasmid;

FIG. 7 is an explanatory diagram illustrating a gene expression method according to a fourth embodiment;

FIG. 8 is an explanatory diagram illustrating one example of a conventionally known gene expression method;

FIG. 9 is an explanatory diagram illustrating the schematic configuration of a plasmid for homologous recombination of Escherichia coli genome;

FIG. 10A is an explanatory diagram illustrating primers used for cloning of DNA fragments;

FIG. 10B is an explanatory diagram illustrating primers used for cloning of DNA fragments;

FIG. 11 is an explanatory diagram illustrating the schematic configuration of a plasmid for homologous recombination of Escherichia coli genome;

FIG. 12A is an explanatory diagram illustrating primers used for cloning of DNA fragments;

FIG. 12B is an explanatory diagram illustrating primers used for cloning of DNA fragments;

FIG. 13 is an explanatory diagram illustrating a destination vector for level 0;

FIG. 14 is an explanatory diagram illustrating destination vectors for level 1;

FIG. 15 is an explanatory diagram illustrating the state of introducing an excised sequence and the like into the yeast genome;

FIG. 16 is an explanatory diagram illustrating the configurations of level 1 modules used for introduction into the yeast genome; and

FIG. 17 is an explanatory diagram illustrating primers used for cloning of DNA fragments.

### DESCRIPTION OF EMBODIMENTS

### A. First Embodiment

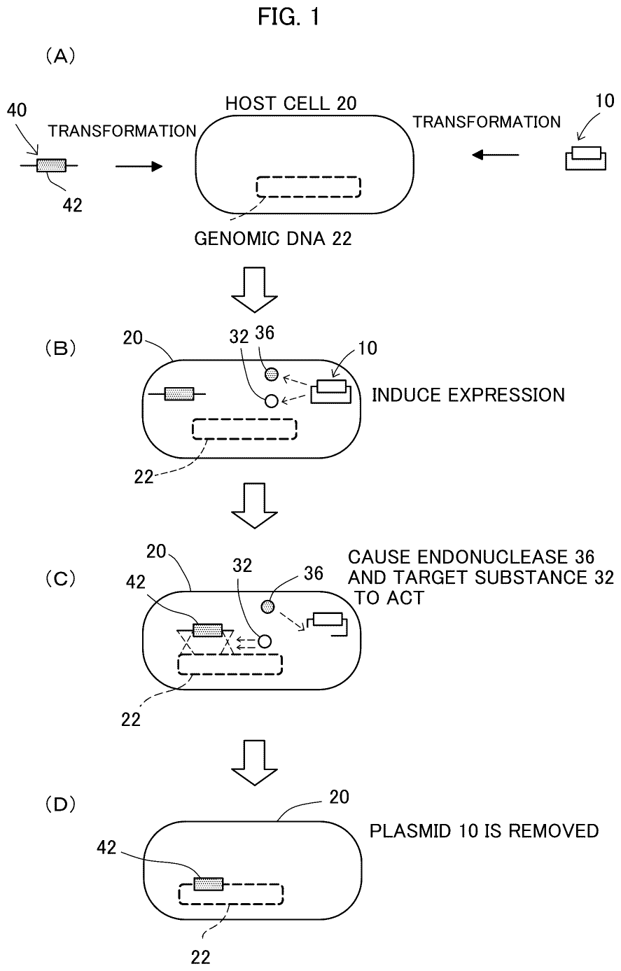

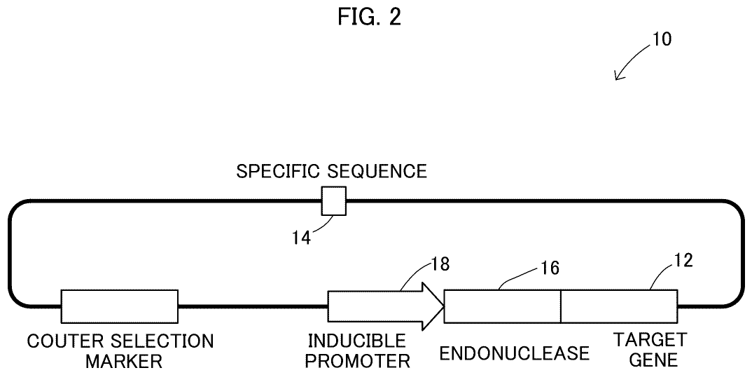

FIG. 1 is an explanatory diagram illustrating a gene expression method according to a first embodiment. FIG. 2 is an explanatory diagram illustrating the schematic configuration of a target gene-introducing plasmid 10 used in the gene expression method.

The gene expression method of this embodiment first provides a target gene-introducing plasmid 10 where a target gene 12 that is to be introduced into and to be expressed in a host cell 20 and an endonuclease gene 16 that specifically recognizes a specific sequence 14 are placed to be expressible under control of a single inducible promoter 18 and where one or more specific sequences 14 are introduced (as shown in FIG. 1(A) and FIG. 2). The gene expression method of this embodiment introduces the target gene-introducing plasmid 10 into the host cell 20 and thereby causes the expression of the target gene 12 included in the target gene-introducing plasmid 10 to be readily controlled on and off at a desired timing in the host cell 20. The configuration of the target gene-introducing plasmid 10 shown in FIG. 2 is described first in detail.

The target gene 12 herein denotes a gene that is introduced into the host cell 20 by using the target gene-introducing plasmid 10 and that is subjected to on-off control of the gene expression at a desired timing to be transiently expressed. The target gene 12 may be, for example, a “gene for modifying a nucleic acid included in the host cell 20”. The “gene for modifying the nucleic acid included in the host cell 20” may be, for example, a “gene for modifying a genomic DNA included in the host cell 20”, a “gene for modifying a plasmid included in the host cell 20” or a “gene for modifying an RNA included in the host cell 20”.

The “gene for modifying the genomic DNA included in the host cell 20” may be, for example, a gene of an enzyme that accelerates a gene recombination reaction. More specifically, the “gene for modifying the genomic DNA included in the host cell 20” may be, for example, a “gene for a homologous recombination reaction”, a “gene for a site-specific recombination reaction” or a “gene relating to genome editing”.

The “gene for the homologous recombination reaction” is a gene of an enzyme that accelerates introduction of a DNA sequence into the genomic DNA and may be, for example, a gene that codes a λ(lambda) phage-derived λ-Red recombinase system, i.e., a gene comprised of exo, bet and gam. In another example, the “gene for the homologous recombination reaction” may be a homologous gene of the λ phage-derived recombinase gene described above and a recombinase gene having similar activity (Proc. Natl. Acad. Sci. USA, 105, 1626, 2008). The 2 phage-derived recombinase gene described above or the like is favorably used as the “target gene 10” in a prokaryotic cell, such as Escherichia coli. FIG. 1 illustrates an example of a gene expression method when the “gene for the homologous recombination reaction” is used as the target gene 12.

The “gene for the site-specific recombination reaction” is a gene of an enzyme that accelerates excision of a DNA sequence from the genome or introduction of a DNA sequence into the genome. A variety of genes listed below are examples of the “gene for the site-specific recombination reaction” used regardless of whether the cell is a prokaryotic cell or a eukaryotic cell. One example is a Cre gene for the site-specific recombination reaction in a Cre-loxP recombination system using Cre recombinase and loxP sequences. Not only the system using the Cre recombinase but a system using, for example, any of FLP recombinase, R recombinase and Dre recombinase, which all belong to a bidirectional tyrosine subfamily like the Cre recombinase, is similarly used for a wide variety of organism species. Among the recombinases belonging to the bidirectional tyrosine subfamily, for example, the respective genes of Dre, VCre, SCre, Vika and Nigri recombinases are all homologous genes of the Cre recombinase and are known to have substantially similar mechanisms of action to that of the Cre recombinase, irrespective of a difference in target sequence. As in the case of using the Cre recombinase, using the recombinase obtained from such a homologous gene of the Cre recombinase gene is desirable to effectively enhance the efficiency of homologous recombination in the wide variety of organism species (Xueying Tian et. al., JBC REVIEWS, 296, 100509, January 2021). The variety of recombinase genes described above are usable as the “gene for the site-specific recombination reaction”

Examples of the “gene relating to genome editing” are genes configuring, for example, ZFN, TALEN, and CRISPR-Cas9 systems. The genome editing technology denotes a technique of cutting a DNA in the vicinity of a target sequence to activate the repair, introducing a mutation in the course of the repair and accelerating the homologous recombination via the target sequence. For example, a typical CRISPR-Cas9 system is comprised of a guide RNA (gRNA) having a sequence complementary to a target DNA sequence and a protein Cas9 that performs cutting of a DNA. Inducing the transcription of gRNA and the gene expression of Cas9 enables the DNA in the vicinity of the target sequence to be cut.

Another example of the “gene for modifying the genomic DNA included in the host cell 20” is a gene of a transposon-cutting enzyme (transposase). Using transposase allows for recombination of a DNA sequence between specific inverse terminal sequences that are recognizable by transposase. The enzyme for genome editing, such as Cas9, described above is one type of nucleases. The target gene 12 used may, however, be any of genes of nucleases other than those for genome editing: for example, nickase that cuts only one strand of a DNA to insert a nick; exonucleases that are enzymes sequentially cutting nucleotides from the end of a nucleic acid; and endonucleases (including restriction enzymes) that are enzymes cutting a polynucleotide chain within a nucleic acid. The target gene 12 used may also be any of genes of DNA ligases and RNA ligases that serve as a catalyst in formation of a phosphodiester bond of a 5′ phosphate terminal and a 3′ hydroxyl terminal adjacent to each other in a double strand DNA or RNA.

The target gene 12 is, however, not limited to the “gene for modifying the genomic DNA included in the host cell 20” described above but may be other genes. The gene expression method of the present disclosure is applicable to a variety of genes that are desired to be transiently expressed at a desired timing.

The specific sequence 14 included in the target gene-introducing plasmid 10 of the embodiment and the endonuclease gene 16 that specifically recognizes this specific sequence are not specifically limited. There is only a requirement that, when the target gene-introducing plasmid 10 is introduced into the host cell 20 and the endonuclease gene 16 is expressed, the expressed endonuclease cuts the target gene-introducing plasmid 10. Examples of the endonuclease coded by the endonuclease gene 16 include restriction enzymes, homing endonucleases, Cas9 nucleases, meganucleases (MN), zinc finger nucleases (ZFN), transcription activator-like effector nucleases (TALEN) and the like. The homing endonuclease may be an endonuclease coded by an intron (with a prefix of I-) or may be an endonuclease included in an intein (with a prefix of PI-). Concrete examples of the homing endonuclease I-CeuI, I-SceI, I-OnuI, PI-PspI, and PI-SceI. The homing endonucleases generally have longer recognition sequences than those of the restriction enzymes. The homing endonuclease thus desirably has a low probability that the recognition sequence thereof is present on the genome of the host cell and has a higher possibility to specifically cut only the specific sequence 14 included in the target gene-introducing plasmid 10. The Cas9 nucleases, the transcription activator-like effector nucleases (TALEN) and the like also have relatively long recognition sequences like the homing endonucleases but have more complicated necessary constituent elements. Accordingly, the homing endonuclease is preferable in terms of reducing the size of the target gene-introducing plasmid 10 and achieving the compact configuration.

The specific sequence 14 may be appropriately set according to the endonuclease coded by the endonuclease gene 16, as a sequence that is specifically recognized by the endonuclease. One or more specific sequences 14 are introduced into the target gene-introducing plasmid 10. FIG. 2 illustrates the target gene-introducing plasmid 10 including one specific sequence 14 as one example. According to the embodiment, the specific sequence 14 that is specifically recognized by the endonuclease, and the cutting location by the endonuclease are placed at different locations from the target gene 12, the endonuclease gene 16 and the inducible promoter 18.

The inducible promoter 18 included in the target gene-introducing plasmid 10 of the embodiment is only required to have a function of inducing expression under specific conditions and is not specifically limited. The inducible promoter 18 is, for example, a promoter that induces expression in the presence of a specific substance, a promoter that induces expression under a specific temperature condition, a promoter that induces expression in response to a variety of stresses, or a promoter that induces expression by specific light. The inducible promoter 18 used may be appropriately selected, for example, according to the type of the host cell 20. Concrete examples of the inducible promoter 18 include galactose inducible promoters such as a GAL promoter (GAL1/GAL10 promoter), Tet-on/Tet-off system promoters that perform induction by addition or removal of tetracycline or a derivative thereof, and gene promoters that code heat shock proteins (HSP), such as HSP10, HSP60, and HSP90. In another example, a CUP1 promoter that is activated by addition of copper ion may be used as the inducible promoter 18. In the case where the host cell is a prokaryotic cell, such as Escherichia coli, the inducible promoter 18 may be a lac promoter induced by IPTG, a cspA promoter induced by a cold shock or an araBAD promoter induced by arabinose.

In the target gene-introducing plasmid 10 of the embodiment, the target gene 12 and the endonuclease gene 16 are placed to be expressible under the control of the single inducible promoter 18 as described above. In the case where a prokaryotic cell, such as Escherichia coli, is used as the host cell 20, the target gene 12 and the endonuclease gene 16 are to be placed in series downstream of the single inducible promoter 18 as shown in FIG. 2. In this case, the inducible promoter 18, the target gene 12 and the endonuclease 16 function as an operon, and the target gene 12 and the endonuclease gene 16 are both transcribed into mRNA to be expressed by induction of the inducible promoter 18. According to the embodiment, the endonuclease gene 16 is placed on an upstream side that generally has a higher expression level, and the target gene 12 is placed on a downstream side. The opposite arrangement is, however, also allowable.

In the case where a eukaryotic cell is used as the host cell 20, a bidirectional promoter is used as the inducible promoter 18, and the target gene 12 and the endonuclease gene 16 are to be placed on respective sides of the bidirectional promoter to sandwich the bidirectional promoter and are to be connected with each other to be expressible at the time of induction of the bidirectional promoter. The bidirectional promoter is, for example, the GAL promoter (GAL1/GAL10 promoter). The GAL promoter is a sequence located between GAL1 and GAL10 of budding yeast. Using this promoter allows for simultaneous induction of the transcription of two genes bidirectionally.

In the case where a eukaryotic cell is used as the host cell 20, the target gene 12 and the endonuclease gene 16 may be connected with each other by means of a linker to be controlled by one single inducible promoter 18 and to be simultaneously transcribed. Such an expression system is called bicistronic expression, and an internal ribosome entry site (IRES) or a 2A peptide may be used as the linker for connecting the target gene 12 and the endonuclease gene 16 with each other. Using such a linker allows for continuous transcription from an ORF of a gene on an upstream side to an ORF of a gene on a downstream side across the linker and enables the two genes to be expressed simultaneously.

The target gene-introducing plasmid 10 of the embodiment may further include a marker gene. Examples of the marker gene include drug-resistant marker genes, auxotrophic marker genes, and counter selection marker genes. Inclusion of such a marker gene allows for efficient selection of the host cell with the target gene-introducing plasmid 10 introduced therein. FIG. 2 illustrates the target gene-introducing plasmid 10 including a counter selection marker gene as one example.

The target gene-introducing plasmid 10 of the embodiment described above may be produced, based on a variety of conventionally known and available plasmids. Examples of this plasmid include: YCp-type Escherichia coli-yeast shuttle vectors, such as pRS413, pRS414, pRS415, pRS416, YCp50, pAUR112 and pAUR123; YEp-type Escherichia coli-yeast shuttle vectors, such as pYES2 and YEp 13; YIp-type Escherichia coli-yeast shuttle vectors, such as pRS403, pRS404, pRS405, pRS406, pAUR101 and pAUR135; Escherichia coli-derived plasmids (for example, ColE plasmids such as pBR322, pBR325, pUC18, pUC19, pUC118, pUC119, pTV118N, pTV119N, pBluescript, pHSG298, pHSG396 and pTrc99A; p 15A plasmids such as pACYC177 and pACYC184; and pSC101 plasmids such as pMW118, pMW119, pMW218 and pMW219), Agrobacterium-derived plasmids (for example, pBI101), and Bacillus subtilis-derived plasmids (for example, pUB110 and pTP5).

The gene expression method of the embodiment provides the target gene-introducing plasmid 10 described above and subsequently uses this target gene-introducing plasmid 10 to lead to transformation of the host cell 20 as shown in FIG. 1(A). Either a prokaryotic cell or a eukaryotic cell may be used as the host cell 20. Examples of the prokaryotic cell include bacteria, such as Escherichia coli and Bacillus subtilis. Examples of the eukaryotic cell include fungi such as filamentous fungi and yeasts, plant cells and animal cells.

A method of introducing the target gene-introducing plasmid 10 into the host cell 20 is not specifically limited, but any of various known transformation methods may be adopted. For example, the transformation method may be appropriately selected among a calcium chloride method, a competent cell method, a protoplast method, a spheroplast method, or an electric pulse method.

FIG. 1 illustrates one example of the gene expression method of the embodiment using the “gene for the homologous recombination reaction”, such as the λ phage-derived recombinase gene as the target gene 12. In FIG. 1(A), in addition to the target gene-introducing plasmid 10, a vector for genome introduction 40 including a genome introducing sequence 42 to be introduced into a genomic DNA 22 of the host cell 20 is accordingly further introduced into the host cell 20.

The genome introducing sequence 42 included in the vector for genome introduction 40 may be appropriately selected according to the purpose for introducing a nucleic acid fragment into the genomic DNA 22. The genome introducing sequence 42 may be configured by, for example, a base sequence that codes a specific protein, a base sequence in a transcription regulatory region of a promoter, an enhancer, a terminator or the like, a base sequence that codes a transfer RNA (tRNA), a ribosome RNA (RNA), or the like, any of a variety of base sequences of marker genes and the like, or a base sequence as a combination of at least parts of the foregoing.

A pair of homologous sequences are placed at respective ends of the genome introducing sequence 42 to cause homologous recombination to proceed between the homologous sequences and the genomic DNA 22 of the host cell 20. These homologous sequences and a recombination region in the genomic DNA 22 of the host cell 20 are required to have such a high degree of sequence homology as to achieve homologous recombination. The degree of sequence homology between the above homologous sequences and the recombination region in the genomic DNA 22 is, for example, not lower than 60%, is preferably not lower than 80%, is more preferably not lower than 90% and is furthermore preferably not lower than 95%. The pair of homologous sequences described above may have an identical length or may have different lengths. The lengths of these homologous sequences are, for example, not less than 0.02 kb and are preferably not less than 0.05 kb. The lengths of these homologous sequences are also not greater than 3 kb and are preferably not greater than 2 kb. In the case where the genome introducing sequence 42 described above is a base sequence that codes a specific protein, the genome introducing sequence 42 should be incorporated into the genomic DNA 22 in such a state that is expressible within the host cell 20 by homologous recombination using the above homologous sequences.

The gene expression method of the embodiment introduces the target gene-introducing plasmid 10 and the vector for genome introduction 40 into the host cell 20 as shown in FIG. 1(A). The gene expression method subsequently cultures the host cell 20 under an inducing condition of the inducible promoter 18 included in the target gene-introducing plasmid 10 (FIG. 1(B)). This causes the target gene 12 to be expressed and generate a recombinase 32, while causing the endonuclease gene 16 to be expressed and generate an endonuclease 36.

The recombinase 32 generated as described above serves to accelerate a homologous recombination reaction between the genome introducing sequence 42 included in the vector for genome introduction 40 and the genomic DNA 22 and thereby to cause the genome introducing sequence 42 to be incorporated into the genomic DNA 22. Furthermore, the endonuclease 36 generated as described above serves to recognize the specific sequence 14 included in the target gene-introducing plasmid 10 and to cut the target gene-introducing plasmid 10 (FIG. 1(C)). The target gene-introducing plasmid 10 is cut by the endonuclease 36, so as to be removed from the host cell 20. This accordingly provides the host cell 20 with the genome introducing sequence 42 incorporated into the genomic DNA 22 thereof without including the target gene-introducing plasmid 10 (FIG. 1(D)).

The gene expression method of the embodiment configured as described above introduces the target gene-introducing plasmid 10 where the target gene 12 and the endonuclease gene 16 are placed to be expressible under control of the single inducible promoter 18 and where the specific sequence 14 is introduced, into the host cell 20 and cultures the host cell 20 under the inducing condition of the inducible promoter 18. This causes the expression of the target gene 12 and of the endonuclease gene 16 and causes the expressed endonuclease 36 to cut the specific sequence 14 and thereby remove the target gene-introducing plasmid 10 from the host cell 20. This method accordingly enables the target gene 12 to be expressed at a desired timing by a simple operation of introducing the target gene-introducing plasmid 10 into the host cell 20 and inducing the inducible promoter 18, and subsequently causes the target gene-introducing plasmid 10 to be removed and make the expression of the target gene 12 off, without requiring any further operation.

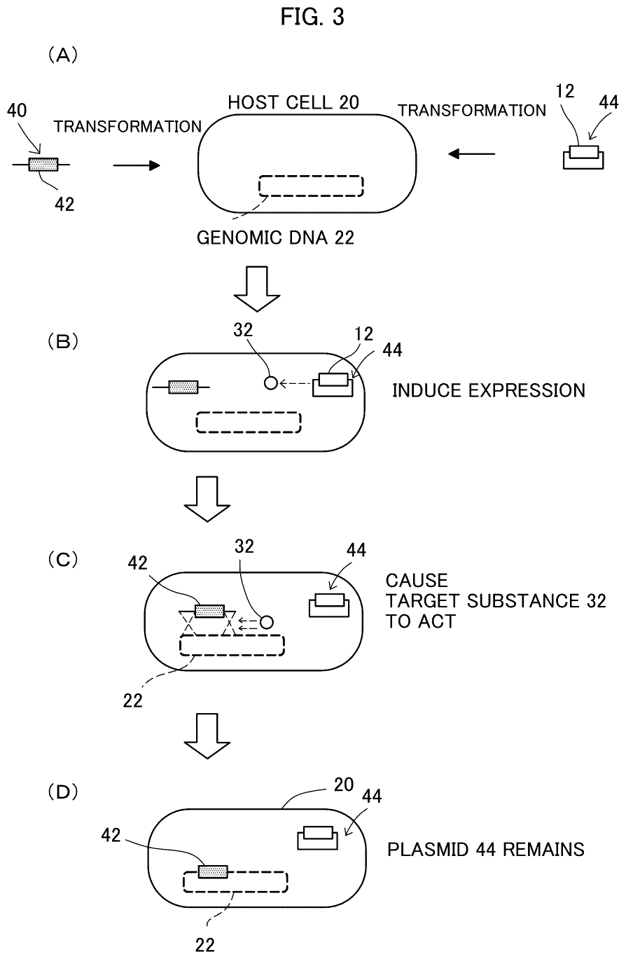

FIG. 3 is an explanatory diagram illustrating one example of a conventionally known gene expression method that is different from the gene expression method of the embodiment but uses a target gene-introducing plasmid 44 in place of the target gene-introducing plasmid 10. The target gene-introducing plasmid 44 does not include the endonuclease gene 16 but otherwise has a similar configuration to that of the target gene-introducing plasmid 10 shown in FIG. 2. The gene expression method shown in FIG. 3 introduces the target gene-introducing plasmid 44 and a vector for genome introduction 40 into a host cell 20 (FIG. 3(A)) and cultures the host cell 20 under the inducing condition of the inducible promoter 18, so as to cause expression of a target gene 12 and to generate a recombinase 32 (FIG. 3(B)). As a result, this accelerates a homologous recombination reaction between a genome introducing sequence 42 included in the vector for genome introduction 40 and a genomic DNA 22 and causes the genome introducing sequence 42 to be incorporated into the genomic DNA 22 (FIG. 3(C)). Even after conclusion of the homologous recombination reaction described above, however, the target gene-introducing plasmid 44 remains in the host cell 20 (FIG. 3(D)). It is thus desired to remove the target gene-introducing plasmid 44 after conclusion of a desired homologous recombination reaction, in order to suppress further expression of the target gene 12 included in the remaining target gene-introducing plasmid 44 and the resulting progress of an undesired reaction within the host cell 20. In order to achieve this demand, however, there is, for example, a need for cutting the target gene-introducing plasmid 44 by counter selection or by the endonuclease. This complicates the entire operations of the gene expression method. The gene expression method of the embodiment, on the other hand, does not require any further operation for removing the target gene-introducing plasmid 44 and thereby simplifies the entire operations of the gene expression method.

As described above, the gene expression method of the embodiment is an excellent method that is only required to introduce the target gene-introducing plasmid 10 into the host cell 20 and to induce the inducible promoter 18, in order to control on the expression of the target gene 12 induced by the inducible promoter 18 and to subsequently automatically control off the expression of the target gene 12 without requiring any further operation. Many of conventionally known inducible promoters are generally allowed to either control on or control off the expression of a control target gene. Even when an inducible promoter is allowed to perform bidirectional control, there is only a slow rate of shift from the on state to the off state. There has accordingly been no known inducible promoters that allow for an automatic changeover from the on state to the off state.

Using the target gene-introducing plasmid 10 as in the case of the gene expression method of the embodiment causes the endonuclease gene 16 and the target gene 12 to be simultaneously transcribed and translated when the host cell 20 is cultured under the inducing condition of the inducible promoter 18. This causes the timing of action of the substance (the recombinase 32) obtained by the expression of the target gene 12 to be substantially equal to the timing of action of the endonuclease 36 obtained by the expression of the endonuclease gene 16. As a result, after the action of the substance obtained by the expression of the target gene 12 (after the homologous recombination by the recombinase 32), this causes the target gene-introducing plasmid 10 to be promptly cut and removed and thereby makes the expression of the target gene 12 off.

As described above, after the action of the substance obtained by the expression of the target gene 12, the gene expression method of the embodiment relatively strictly controls off the expression of the target gene 12 within a relatively short time period. Accordingly, the gene expression method of the embodiment is especially useful when the target gene 12 used is a gene that is likely to be expressed at an undesired timing and to show the toxicity in the host cell 20. More specifically, the gene expression method of the embodiment is useful when the target gene 12 used is, for example, a “gene that modifies (including epigenetic modifications) the genomic DNA included in the host cell 20”. In such cases, when a plasmid including the target gene 12 remains within the host cell 20 and causes the target gene 12 to be expressed, this is likely to have adverse effects on expression of proteins and cell division, which are basic vital activities. The gene expression method of the embodiment, however, enables the unwanted target gene-introducing plasmid 10 to be automatically and promptly removed and thereby readily reduces the possible disadvantages described above.

### B. Second Embodiment

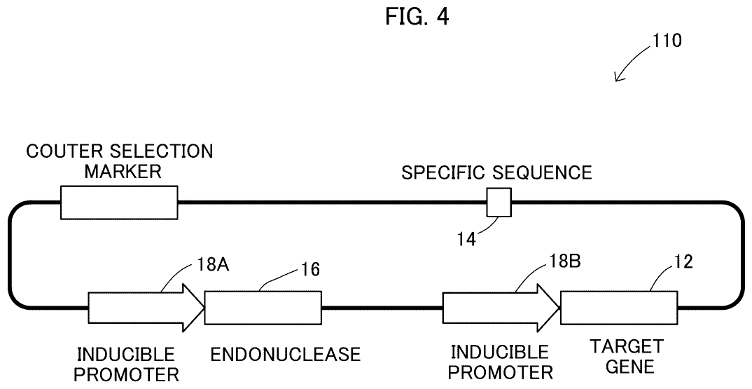

FIG. 4 is an explanatory diagram illustrating the schematic configuration of a target gene-introducing plasmid 110 according to a second embodiment. The target gene-introducing plasmid 110 may be used in place of the target gene-introducing plasmid 10, in the gene expression method of the first embodiment shown in FIG. 1.

According to the first embodiment, the target gene 12 and the endonuclease gene 16 are induced by the single inducible promoter 18. A different configuration may, however, be applied. In the target gene-introducing plasmid 110 of the second embodiment, individual inducible promoters are provided to respectively induce the target gene 12 and the endonuclease gene 16. More specifically, the target gene-introducing plasmid 110 includes an inducible promoter 18A that induces the endonuclease gene 16 and an inducible promoter 18B that induces the target gene 12. The inducible promoters 18A and 18B denote promoters that induce simultaneous expression and that cause the endonuclease gene 16 and the target gene 12 to be simultaneously expressed. In one example, the inducible promoters 18A and 18B may be identical inducible promoters. In another example, the inducible promoters 18A and 18B may be a combination of inducible promoters that show the same inductivity but do not have fully identical sequences, for example, one of the inducible promoters has a homologous sequence of the other or has a displaced sequence from the other.

As described above, using the target gene-introducing plasmid 110 that includes one or more inducible promoters to simultaneously induce the expression of the endonuclease gene 16 and the expression of the target gene 12 has similar advantageous effects to those of the first embodiment. For example, in the case where the target gene 12 is comprised of a plurality of genes, individual inducible promoters may be provided to simultaneously induce the expression of the respective genes of the target gene 12.

### B. Third Embodiment

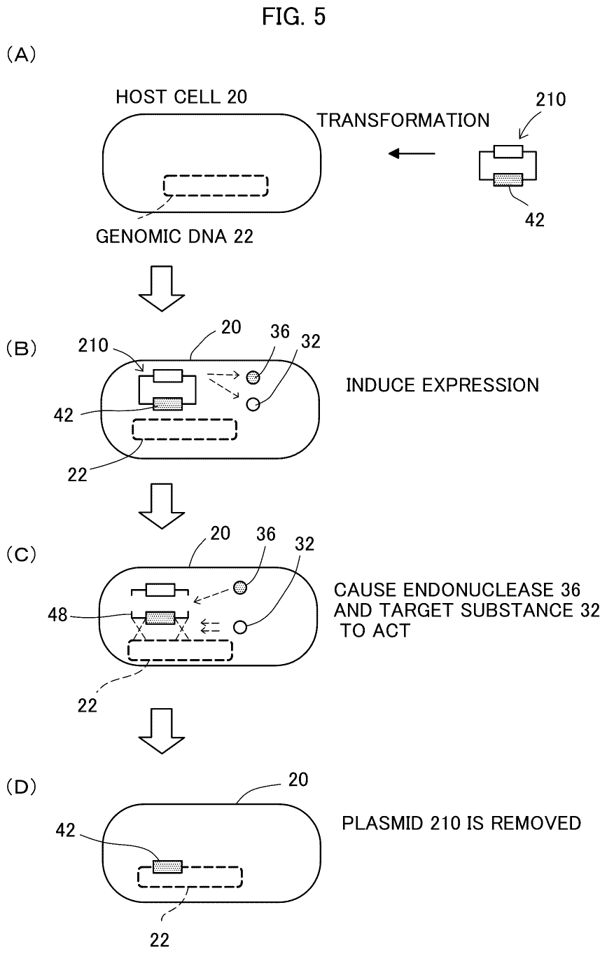

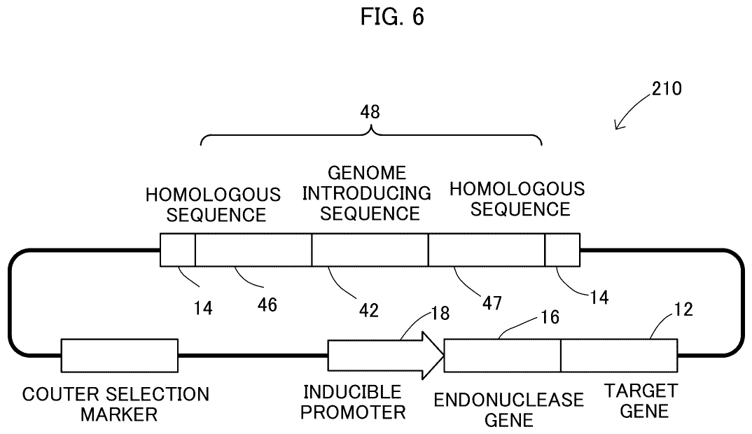

FIG. 5 is an explanatory diagram illustrating a gene expression method according to a third embodiment. FIG. 6 is an explanatory diagram illustrating the schematic configuration of a target gene-introducing plasmid 210 used in this gene expression method. In the description of the third embodiment, constituent elements similar to those of the first embodiment are expressed by similar reference signs.

The gene expression method of the third embodiment uses the target gene-introducing plasmid 210, in place of the target gene-introducing plasmid 10 used in the gene expression method of the first embodiment. As shown in FIG. 6, the target gene-introducing plasmid 210 includes an inducible promoter 18, an endonuclease gene 16, and a target gene 12, like the target gene-introducing plasmid 10. The target gene-introducing plasmid 210 further includes a nucleic acid sequence where a specific sequence 14, a homologous sequence 46, a genome introducing sequence 42, a homologous sequence 47 and a specific sequence 14 are placed in this order, in place of the single specific sequence 14 included in the target gene-introducing plasmid 10. The genome introducing sequence 42 is a sequence to be introduced into a genomic DNA 22 of the host cell 20, like the genome introducing sequence 42 of the first embodiment. The homologous sequences 46 and 47 are sequences homologous with a sequence at a location in the genomic DNA 22 of the host cell 20 where the genome introducing sequence 42 is to be introduced, and are sequences used for homologous recombination with the genomic DNA 22 at the time of introducing the genome introducing sequence 42 into the host cell 20. The third embodiment accordingly uses a plasmid having two specific sequences 14 and further including a sequence that is introduced in between these two specific sequences 14 and that allows for homologous recombination with the genomic DNA 22 of the host cell 20, as the target gene-introducing plasmid 210. The target gene-introducing plasmid 210 may further include another specific sequence 14 at another location. The target gene-introducing plasmid 210 is thus required to have two or more specific sequences 14 including the two specific sequences 14 described above.

The gene expression method of the third embodiment first causes transformation of a host cell 20 with the target gene-introducing plasmid 210 (FIG. 5(A)) and cultures the host cell 20 under an inducing condition of the inducible promoter 18 (FIG. 5(B)). This causes expression of the target gene 12 to generate a recombinase 32, while causing expression of the endonuclease gene 16 to generate an endonuclease 36.

The endonuclease 36 generated as described above serves to recognize the pair of specific sequences 14 included in the target gene-introducing plasmid 210 and to cut the target gene-introducing plasmid 210 at two locations (FIG. 5(C)). As shown in FIG. 6, according to the third embodiment, the pair of specific sequences 14 are placed across the sequence including the homologous sequences 46 and 47 and the genome introducing sequence 42. The endonuclease 36 accordingly serves to excise the sequence including the homologous sequences 46 and 47 and the genome introducing sequence 42, from the target gene-introducing plasmid 210. The excised sequence including the homologous sequences 46 and 47 and the genome introducing sequence 42 is referred to as “recombination sequence 48” (as shown in FIG. 6). Furthermore, the recombinase 32 generated as described above serves to accelerate a homologous recombination reaction between the recombination sequence 48 excised from the target gene-introducing plasmid 210 and the genomic DNA 22 and thereby to cause the genome introducing sequence 42 to be incorporated into the genomic DNA 22. Furthermore, the endonuclease 36 serves to cut the target gene-introducing plasmid 210 as described above, so that the target gene-introducing plasmid 210 is removed from the host cell 20. As a result, this provides the host cell 20 with the genome introducing sequence 42 incorporated into the genomic DNA 22 thereof without including the target gene-introducing plasmid 210 (FIG. 5(D)).

Like the first embodiment, the configuration of this embodiment readily causes the target gene 12 to be expressed at a desired timing and subsequently causes the target gene-introducing plasmid 210 to be removed from the host cell 20 and makes the expression of the target gene 12 off by the simple operation of introducing the target gene-introducing plasmid 210 into the host cell 20 and inducing the inducible promoter 18. Furthermore, according to the third embodiment, the target gene-introducing plasmid 210 includes the genome introducing sequence 42 in addition to the target gene 12, and the endonuclease 36 serves to cut the target gene-introducing plasmid 210 and excise the recombination sequence 48 including the genome introducing sequence 42. The configuration of the third embodiment accordingly does not require the operation of separately providing the vector for genome introduction 40 to cause transformation of the host cell 20 unlike the first embodiment and simplifies the entire operations for introducing a nucleic acid fragment into the genomic DNA 22. For example, in the case where a prokaryotic cell is used as the host cell 20 and a λ phage-derived recombinase gene is used as the target gene 12, homologous recombination of the genomic DNA is enabled by a simple operation (a single transformation) equivalent to transformation of Escherichia coli with a plasmid. The target gene-introducing plasmid 210 of the third embodiment shown in FIG. 6 has the single inducible promoter 18, like the target gene-introducing plasmid 10 of the first embodiment. According to a modification, however, the endonuclease gene 16 and the target gene 12 may be induced by individual inducible promoters, like the second embodiment. Expression of the endonuclease gene 16 and the target gene 12 under control of one or more inducible promoters which induce simultaneous expression, has similar advantageous effects.

### C. Fourth Embodiment

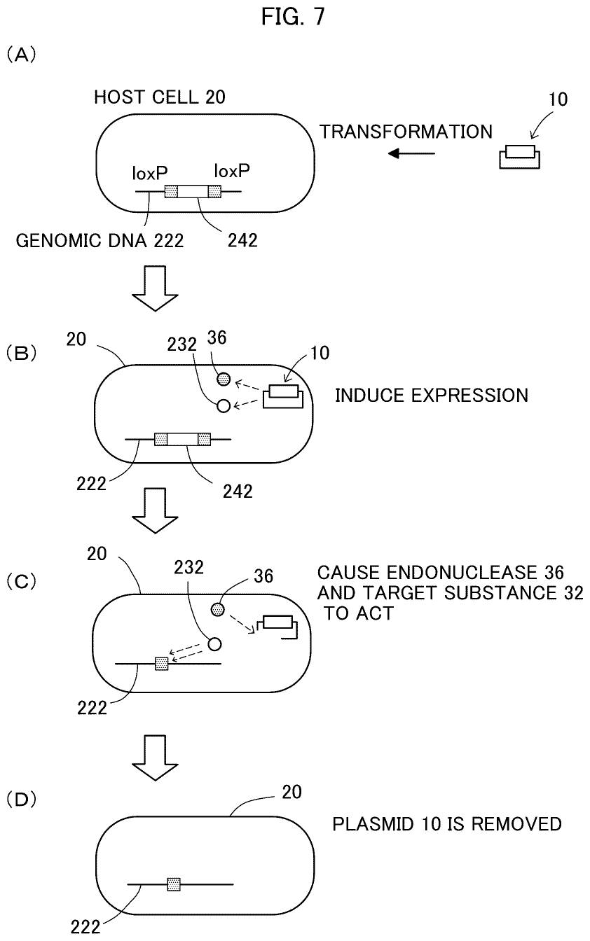

FIG. 7 is an explanatory diagram illustrating a gene expression method according to a fourth embodiment. In the description of the fourth embodiment, constituent elements similar to those of the first embodiment are expressed by similar reference signs.

The first embodiment shown in FIG. 1 and FIG. 2 and the third embodiment shown in FIG. 5 and FIG. 6 use the gene that expresses the recombinase 32 as the target gene 12 and causes the genome introducing sequence 42 to be incorporated into the genomic DNA 22 by homologous recombination between the genome introducing sequence 42 and the genomic DNA 22 using the homologous sequence. The gene expression method of the fourth embodiment, on the other hand, uses a gene of an enzyme that accelerates a site-specific recombination reaction, as the target gene 12 included in the target gene-introducing plasmid 10, to cut out an “excised sequence 242” from a genomic DNA 222 of a host cell 20. As one example, FIG. 7 illustrates a site-specific recombination reaction in a Cre-loxP recombination system using Cre recombinase and loxP sequences. More specifically, in the genomic DNA 222, loxP sequences are placed at respective ends of the excised sequence 242. The target gene-introducing plasmid 10 having a configuration similar to that shown in FIG. 2 includes a Cre recombinase gene as the target gene 12.

The gene expression method of the fourth embodiment causes transformation of a host cell 20 with the target gene-introducing plasmid 10 (FIG. 7(A)) and cultures the host cell 20 under the inducing condition of the inducible promoter 18, so as to cause expression of the target gene 12 to generate a Cre recombinase 232, while causing expression of the endonuclease gene 16 to generate an endonuclease 36 (FIG. 7(B)). The Cre recombinase 232 generated as described above serves to cause a site-specific recombination reaction to proceed in the genomic DNA 222 and to cut out the excised sequence 242. Furthermore, the endonuclease 36 generated as described above serves to recognize a specific sequence 14 included in the target gene-introducing plasmid 10 and to cut the target gene-introducing plasmid 10 (FIG. 7(C)). This causes the target gene-introducing plasmid 10 to be removed from the host cell 20. As a result, this provides the host cell 20 after excision of the excised sequence 242 from the genomic DNA 222 thereof without including the target gene-introducing plasmid 10 (FIG. 7(D)).

Like the first embodiment, the configuration of this embodiment readily causes the target gene 12 to be expressed at a desired timing and subsequently causes the target gene-introducing plasmid 10 to be removed from the host cell 20 and makes the expression of the target gene 12 off by the simple operation of introducing the target gene-introducing plasmid 10 into the host cell 20 and inducing the inducible promoter 18.

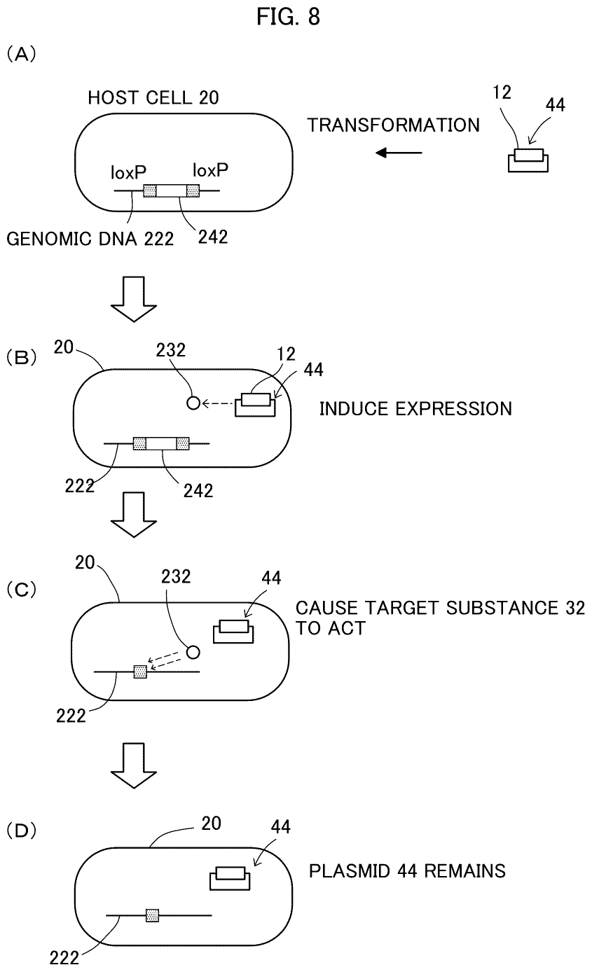

FIG. 8 is an explanatory diagram illustrating one example of a conventionally known gene expression method that is different from the gene expression method of the fourth embodiment but uses a target gene-introducing plasmid 44 in place of the target gene-introducing plasmid 10. The target gene-introducing plasmid 44 does not include the endonuclease gene 16 but otherwise has a similar configuration to that of the target gene-introducing plasmid 10 of the fourth embodiment. The gene expression method shown in FIG. 8 introduces the target gene-introducing plasmid 44 into a host cell 20 (FIG. 8(A)) and cultures the host cell 20 under the inducing condition of the inducible promoter 18, so as to cause expression of a target gene 12 and to generate the Cre recombinase 232 (FIG. 8(B)). As a result, this causes a site-specific recombination reaction to proceed in the genomic DNA 222 and cuts out the excised sequence 242 (FIG. 8(C)). Even after conclusion of the site-specific recombination reaction described above, however, the target gene-introducing plasmid 44 remains in the host cell 20 (FIG. 8(D)). It is thus desired to remove the target gene-introducing plasmid 44 after conclusion of a desired site-specific recombination reaction, in order to suppress further expression of the target gene 12 included in the remaining target gene-introducing plasmid 44 and the resulting progress of an undesired reaction within the host cell 20. This complicates the entire operations of the gene expression method. The gene expression method of the fourth embodiment, on the other hand, does not require any further operation for removing the target gene-introducing plasmid 44 and thereby simplifies the entire operations of the gene expression method.

In the fourth embodiment, the target gene-introducing plasmid 10 having the configuration shown in FIG. 2 may be replaced by a target gene-introducing plasmid 110 that has the configuration shown in FIG. 4 and that causes the endonuclease gene 16 and the target gene 12 to be expressed under control of individual inducible promoters. Expression of the endonuclease gene 16 and the target gene 12 under control of one or more inducible promoters which induce simultaneous expression, has similar advantageous effects.

### EXAMPLES

The following describes the present disclosure more specifically by referring to some examples. The present disclosure is, however, not limited to the description of these examples. The examples described below sequentially show the results of adopting the gene expression method described above with reference to FIG. 5 to introduce and express the “gene for the homologous recombination reaction” by using a prokaryotic cell, Escherichia coli, as the host cell 20 and the results of adopting the gene expression method described above with reference to FIG. 7 to introduce and express the “gene for the site-specific recombination reaction” by using a eukaryotic cell, Saccharomyces cerevisiae, as the host cell 20.

<Production of Target Gene-Introducing Plasmid for Homologous Recombination of Escherichia coli Genome>

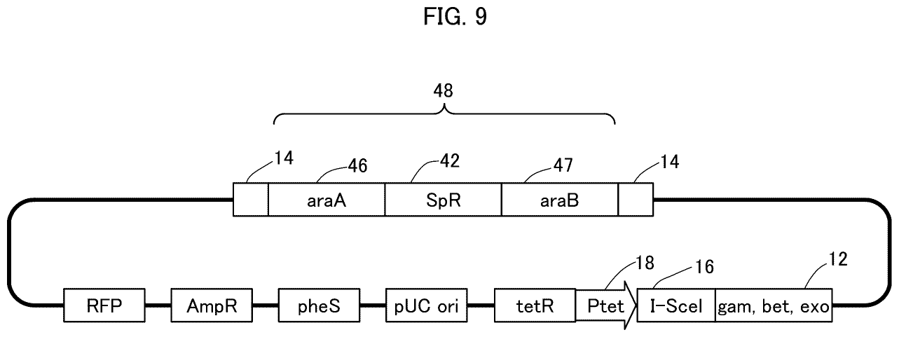

FIG. 9 is an explanatory diagram illustrating the schematic configuration of a target gene-introducing plasmid for homologous recombination of Escherichia coli genome. The target gene-introducing plasmid shown in FIG. 9 is used as the target gene-introducing plasmid 210 in the gene expression method of FIG. 5. Portions of the target gene-introducing plasmid of FIG. 9 corresponding to the respective portions of the target gene-introducing plasmid 210 shown in FIG. 6 are expressed by the same reference signs. The target gene-introducing plasmid shown in FIG. 9 includes a spectinomycin (Sp)-resistance gene (SpR) that is a marker for selection of homologous recombination, as the genome introducing sequence 42; 50 bp sequences that are parts of an araA gene and an arab gene, as the pair of homologous sequences 46 and 47 placed across the genome introducing sequence 42 (homologous sequences at introduction sites in the Escherichia coli genome); and sequences in a recognition site of I-SceI homing endonuclease, as the pair of specific sequences 14 placed outside of the homologous sequences 46 and 47.

The target gene-introducing plasmid also includes a tetR gene and a tetA promoter (inducible promoter 18) required for a Tet-On system whose expression is induced by anhydrotetracycline (ATet) (Microb. Biotechnol. 1, 2, 15-30, 2008); I-SceI as the endonuclease gene 16; and a λ recombinase operon (gam, bet, exo) as the target gene 12, which constitute an inducible expression cassette. The target gene-introducing plasmid further includes a gene of mRFP1.1 (RFP) that is a red fluorescent protein (Nat. Biotechnol. 12, 1567, 2004) and an ampicillin-resistant gene (AmpR), which are used as markers for confirming plasmid retention; and a PheS mutated gene that leads to the death of cells in the presence of 4-chloro-D, L-phenylalanine (4CP), as a counter selection marker. Synthetic terminators Bba_B1002 and Bba_B1006 (Nat. Methods 10, 354-60, 2013) are fused with the I-SceI and the λ recombinase operon. A synthetic promoter BbaJ23118 (Anderson promoter collection) and a synthetic terminator BBa_B1004 are fused with the PheS mutated gene. A lac promoter and a λ imm21 phage-derived to terminator are fused with the RFP gene.

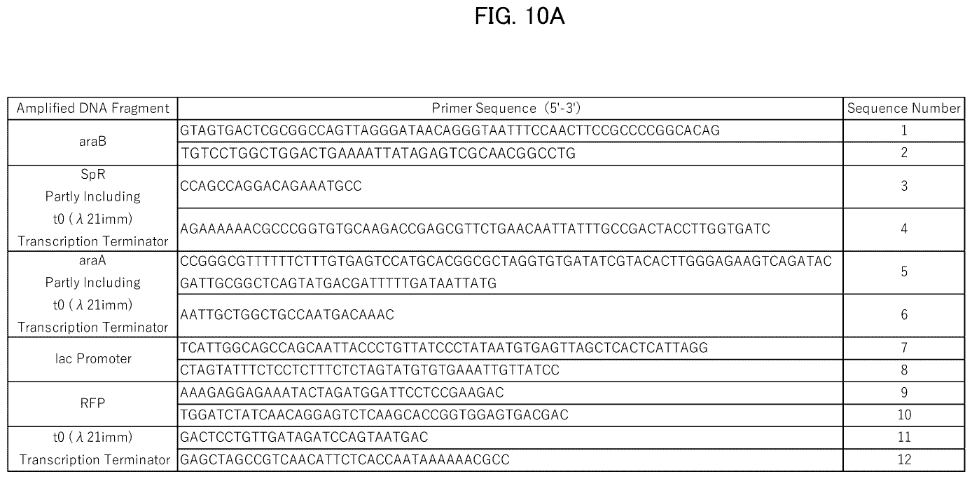

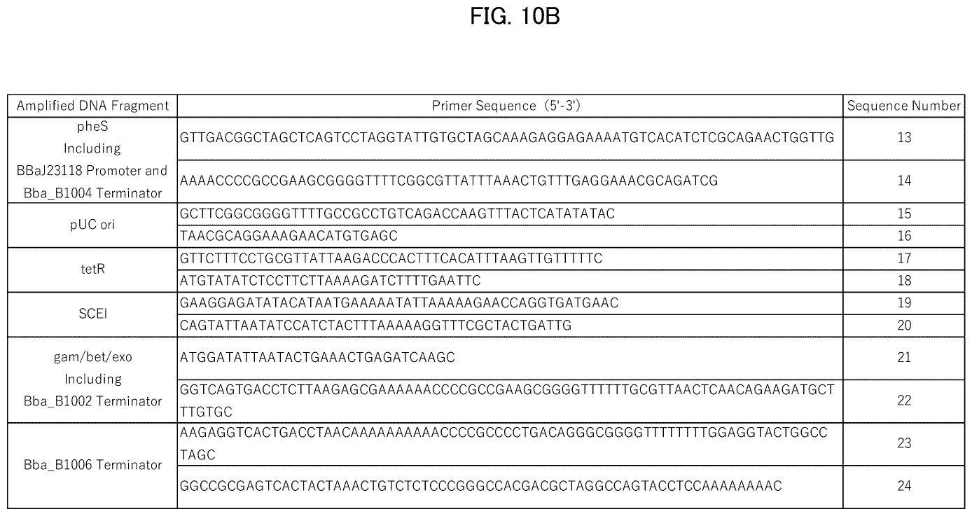

FIG. 10A and FIG. 10B are explanatory diagrams collectively showing primers used for cloning of DNA fragments required for production of the target gene-introducing plasmid described above. The respective “amplified DNA fragments” shown in FIG. 10A and FIG. 10B were amplified by PCR using these primers. A λ phage genomic DNA, a pUC19 plasmid, and a synthetic DNA were used as templates for the amplification. More specifically, an Escherichia coli K12-strain genome was used as the template to amplify a 50 bp fragment from a start of an ORF of the araA gene and a 50 bp fragment from an end of an ORF of the arab gene. A lambda phage genomic DNA was used as the template to amplify gam, bet, and exo genes. The pUC19 plasmid was used as the template to amplify pUC ori. The synthetic DNA was used as the template to amplify SpR (Sci. Rep. 9, 10131, 2019), RFP, and an I-SceI gene (GenBank ID 854590: codon optimized for Escherichia coli), and the tetR gene and the tetA promoter required for the Tet-On system. Each of the primers described above was designed by adding a DNA sequence such as to have an overlap of not less than 15 bp with an adjacent DNA sequence, in order to bind the respective DNA fragments obtained by amplification. The target gene-introducing plasmid shown in FIG. 9 was produced by amplifying the respective target DNA fragments as described above and sequentially binding the DNA fragments using an NEBuilder HiFi DNA Assembly Cloning Kit (manufactured by New England Biolabs) or the like.

### <Transient Expression and Genome Homologous Recombination Reaction of Target Gene>

Plasmid-retaining cells were obtained by transformation of commercially available Escherichia coli (DH5α) competent cells with the target gene-introducing plasmid described above and selection of cells having ampicillin resistance. The transformed plasmid-retaining cells were subjected to liquid culture for 7 hours in a CIRCLEGROW medium (manufactured by MP Biomedicals) containing 100 μg/ml of spectinomycin (Sp) and 50 ng/ml of anhydrotetracycline (Atet) to induce the expression of I-SceI, gam, bet and exo genes, and were subsequently cultured in an LB agar medium containing Sp, Atet and 4-chloro-D, L-phenylalanine (4CP) (hereinafter referred to as “LB agar medium for selection”). The RFP gene is present on the plasmid used for the transformation, so that the plasmid-retaining cells are enabled to detect the fluorescence of RFP. The cells with introduction of an insert (the genome introducing sequence 42: SpR) into the genomic DNA by the action of the recombinase and with removal of the plasmid, on the other hand, have Sp resistance and lose the RFP gene. Culturing these cells in the above “LB agar medium for selection” accordingly provides white colonies. Only the white colonies with removal of the plasmid were thus counted as genome homologous recombinant cells, out of colonies grown in the above “LB agar medium for selection”. The cells after the induction of expression described above were simultaneously cultured in a conventional LB agar medium, and the total number of cultured cells was counted. The purpose of addition of 4CP, which is a selection chemical for counter selection, to the medium, in the process of identifying the genome homologous recombinant cells by the white color of the colonies, is to remove the plasmid with the higher accuracy.

The efficiency of genome homologous recombination and the plasmid retention rate after the above induction of expression in the plasmid-retaining cells were calculated as follows:

Efficiency of genome homologous recombination=the number of white colonies in the LB agar medium for selection/the number of colonies in the LB agar medium

Plasmid retention rate=the number of red colonies in the LB agar medium for selection/the number of colonies in the LB agar medium

The “efficiency of genome homologous recombination” calculated as described above was 1.2×10⁻⁴. No red colonies growing in the LB agar medium for selection were observed, so that the plasmid retention rate after the induction of expression was thought to be less than 1×10⁻⁸. As an example of performing genome homologous recombination by a conventionally known genome homologous recombination method, it has been shown in Proc. Natl. Acad. Sci. USA, 97, 5978, 2000 that the efficiency of genome homologous recombination is approximately 1.0 to 0.1×10⁻⁴. This conventionally known genome homologous recombination method causes the plasmid to remain within the host cell after the homologous recombination as shown in FIG. 3. As described above, the gene expression method of the present disclosure has been proved to provide an equivalent or higher level of the efficiency of genome homologous recombination, compared with the conventionally known genome homologous recombination method and furthermore to automatically remove the plasmid after the induction of expression.

The conventionally known genome homologous recombination method described above requires a longer-time induction for expression of the gene that codes the λ-Red recombinase system subjected to homologous recombination, compared with the gene expression method of the present disclosure. The gene expression method of the present disclosure induces the endonuclease gene 16 along with the target gene 12 for homologous recombination and causes the expressed endonuclease to cut the target gene-introducing plasmid 10. This terminates the induction of expression of the target gene 12 in a relatively short time period. As described above, the gene expression method of the present disclosure induces only the transient expression of the target gene 12 but still has achieved the equivalent or even higher level of the efficiency of genome homologous recombination compared with the conventionally known genome homologous recombination method described above. Achieving the high recombination efficiency by the transient expression system that terminates induction in a short time period, like the configuration of the present disclosure, is unexpectable from the conventional knowledge and findings. In this sense, the gene expression method of the present disclosure provides a novel system that has such significant effects.

Twenty clones of the white colonies in the “LB agar medium for selection” were checked by PCR for the successful genome homologous recombination and were also checked for the sensitivity of ampicillin in order to detect the retention or no retention of the plasmid. As a result, it was confirmed that the homologous recombination was accurately performed and the plasmid was removed with regard to all the strains.

### <Production of Target Gene-Introducing Plasmid for Site-Specific Recombination of Yeast Genome>

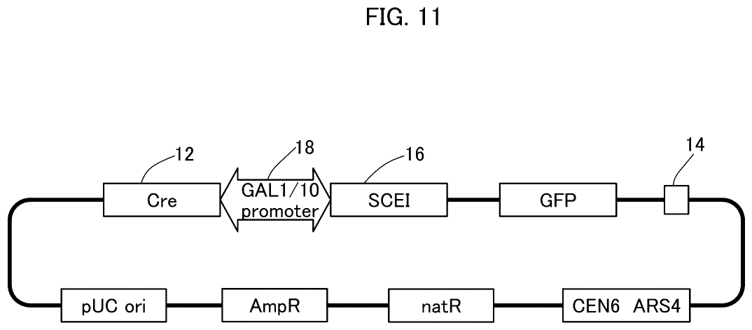

FIG. 11 is an explanatory diagram illustrating the schematic configuration of a target gene-introducing plasmid for site-specific recombination of yeast genome. The target gene-introducing plasmid shown in FIG. 11 is used as the target gene-introducing plasmid 10 in the gene expression method of FIG. 7. Portions of the target gene-introducing plasmid of FIG. 11 corresponding to the respective portions of the target gene-introducing plasmid shown in FIG. 2 are expressed by the same reference signs. The target gene-introducing plasmid shown in FIG. 11 includes a GAL1/10 promoter whose expression is induced by galactose, as a Cre expression-inducing promoter that is the inducible promoter 18, and also includes an I-SceI homing endonuclease gene (SCEI) that is the endonuclease gene 16 and a Cre gene that is the target gene 12, as genes fused with the above promoter. The GAL1/10 promoter is a bidirectional promoter and is accordingly placed between the I-SceI homing endonuclease gene (SCEI) and the Cre gene. The target gene-introducing plasmid further includes a nourseothricin-resistant gene (natR) as a selection marker for yeast, an ampicillin-resistant gene (AmpR) as a plasmid selection marker for Escherichia coli, a gene of AvicFP1 (GFP) that is a green fluorescence protein (PLOS. Biol. 18, e3000936 (2020), and CEN6 and ARS4 that are autonomous replication starting sequences of the yeast.

The Cre gene is fused with a COX5B intron for the purpose of division of a CYC1 terminator and a CDS (Coding Sequence: protein coding region). The SCEI gene is fused with an RPS25A intron for the purpose of division of a CDC53 terminator and a CDS. The GFP gene is fused with a CCW12 promoter and an RPS28A terminator, and a natR gene is fused with a CYC1 promoter and an LEU2 terminator. The target gene-introducing plasmid shown in FIG. 11 includes one recognition site of the I-SceI homing nuclease as the specific sequence 14. The plasmid is cut when the I-SceI homing endonuclease gene is expressed.

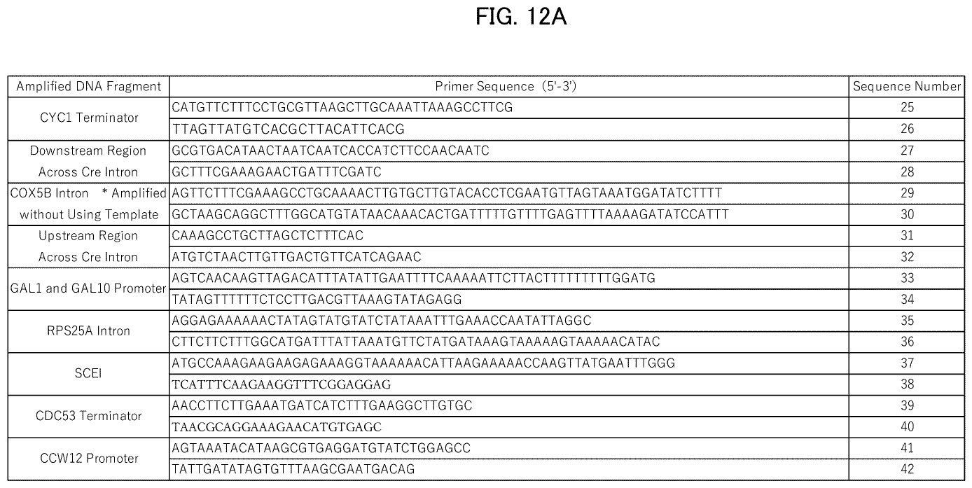

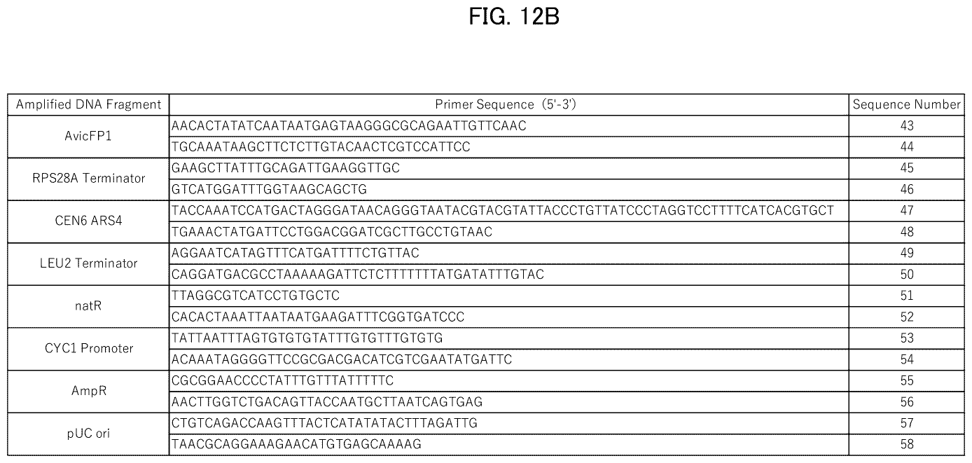

FIG. 12A and FIG. 12B are explanatory diagrams collectively showing primers used for cloning of DNA fragments required for production of the target gene-introducing plasmid described above. The respective “amplified DNA fragments” shown in FIG. 12A and FIG. 12B were amplified by PCR using these primers. A S. cerevisiae BY4742 strain genomic DNA, a pUC19 plasmid, and a synthetic DNA were used as templates for the amplification. More specifically, the S. cerevisiae BY4742 strain genome was used as the template to amplify a CYC1 terminator, a GAL1/10 promoter, an RPS25A intron, a CDC53 terminator, a CCW12 promoter, an RPS28A terminator, an LEU2 terminator, and a CYC1 promoter. The pUC19 plasmid was used as the template to amplify the AmpR gene and a replication origin (ori). The synthetic DNA was used as the template to amplify the Cre gene (GenBank ID AB594821.1), the I-SceI gene (GenBank ID 854590: codon optimized for the yeast genome), the AvicFP1 gene (GenBank ID QEQ56190.1), CEN6 and ARS4 (GenBank ID U03441.1), and the natR gene (GenBank ID S60706.1). Each of the primers described above was designed by adding a DNA sequence such as to have an overlap of not less than 15 bp with an adjacent DNA sequence, in order to bind the respective DNA fragments obtained by amplification. The target gene-introducing plasmid was produced by amplifying the respective target DNA fragments as described above and sequentially binding the DNA fragments using the NEBuilder HiFi DNA Assembly Cloning Kit or the like.

### <Production of Excised Sequence-Introduced Yeast>

In order to perform the gene expression method of FIG. 7 by using the target gene-introducing plasmid shown in FIG. 11, a yeast with the excised sequence 242 placed between a pair of loxP sequences and introduced into the genomic DNA (hereinafter referred to as “excised sequence-introduced yeast”) was produced as the host cell 20 shown in FIG. 7. The following sequentially describes a method of producing the excised sequence-introduced yeast.

### (Production of Cloning Vector)

A variety of cloning vectors described below were produced for production of a Cre/loxP-introduced yeast as the excised sequence-introduced yeast. These vectors were all built by the Golden Gate method (PLOS One 6, e16765, 2011). The Golden Gate method is a known method of stacking genes that uses Type IIS restriction enzyme and T4DNA ligase to insert a plurality of DNA fragments into a vector in a sequence designed in advance. Vectors corresponding to a destination vector for level 0 and destination vectors for level 1 were produced as subcloning vectors required for the Golden Gate method. The following sequentially describes procedures of producing the destination vector for level 0 and the destination vectors for level 1.

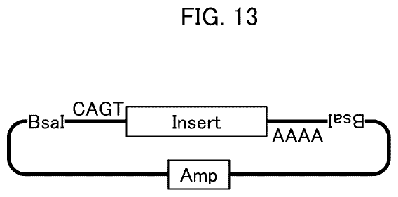

FIG. 13 is an explanatory diagram illustrating a destination vector for level 0. As shown in FIG. 13, the destination vector for level 0 is a vector including a BsaI recognition site. The destination vector for level 0 was produced by amplifying a target DNA fragment by PCR using pUN 19 or a synthetic DNA as the template. A primer used for PCR was artificially synthesized by adding a DNA sequence such as to have an overlap of not less than 15 bp with an adjacent DNA sequence, in order to bind the respective DNA fragments. The destination vector for level 0 was completed by sequentially binding the amplified PCR fragments to one another using the NEBuilder HiFi DNA Assembly Cloning Kit.

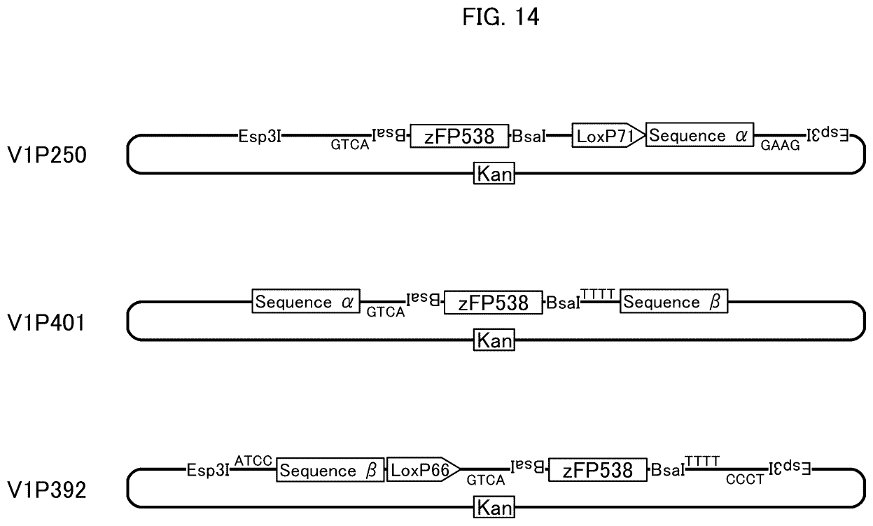

FIG. 14 is an explanatory diagram illustrating destination vectors for level 1. As shown in FIG. 14, the destination vectors for level 1 are a set of multiple different types of vectors. Each of the vectors has a zFP538 gene of a fluorescent protein (Nat. Biotechnol. 17, 969-973 (1999)) and BsaI recognition sites placed in reverse directions across this zFP538 gene. The respective destination vectors for level 1 have different combinations and different layouts including the presence and the absence, of mutated loxP sequences (Plant J., 7, 649-659, 1995), sequences a and B described later, restriction enzyme sites used for the Golden Gate reaction, and cutting locations. These destination vectors for level 1 were produced by amplifying target DNA fragments using pUC19 and a synthetic DNA (including a kanamycin-resistant gene) as the templates and sequentially binding the DNA fragments by using the NEBuilder HiFi DNA Assembly Cloning Kit. The sequences a and B are homologous sequences of 57 bp provided at terminals of the respective amplified fragments, in order to bind the respective amplified fragments obtained by PCR amplification in a desired sequence. Any sequences may be employed for the sequences a and B, as long as the sequences have sufficiently low similarity. Artificial sequences having little similarity with the genome sequence of the yeast were set, and the sequences described in Microb. Cell Fact. 12, 47 (2013) were used as the sequence a and B.

### (Production of Vector Set for Yeast)

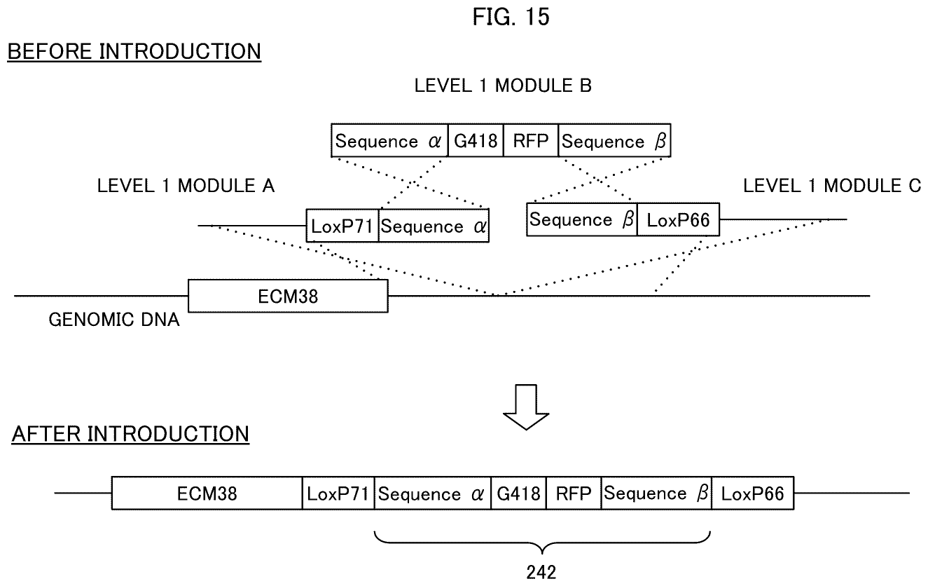

FIG. 15 is an explanatory diagram illustrating the state of introducing the excised sequence 242 (shown in FIG. 7) placed between a pair of loxP sequences into an ECM38 gene locus of the yeast genome by homologous recombination of level 1 modules produced by using the destination vector for level 0 and the destination vectors for level 1 described above. The excised sequence 242 includes a G418-resistant gene (G418 marker) and an RFP gene. As shown in FIG. 15, the vectors for the yeast were designed such that mixing and transformation of a three-fragment set caused recombination between homologous regions (either the sequence a or the sequence B) of the respective fragments and eventually formed one fragment to be introduced into the yeast genome. FIG. 15 illustrates the respective fragments of the three-fragment set used to obtain one fragment to be introduced into the yeast genome are expressed as a level 1 module A, a level 1 module B, and a level 1 module C in a sequence from a 5′-side after binding.

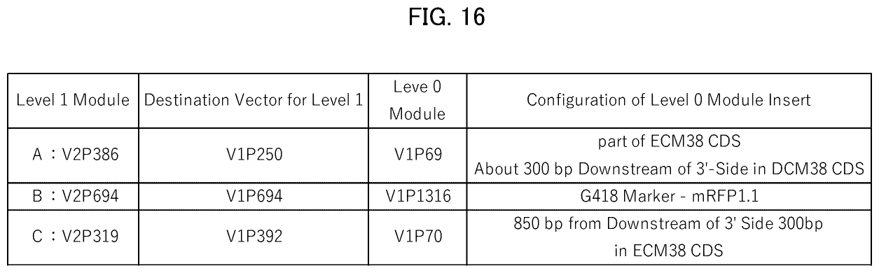

FIG. 16 is an explanatory diagram illustrating the configurations of the respective level 1 modules used for introduction into the yeast genome shown in FIG. 15. More specifically, FIG. 16 collectively shows the type of a destination vector for level 1 used (shown in FIG. 14), the type of a level 0 module, and the configuration of a level 0 module insert with regard to each of the level 1 modules. In FIG. 16, a level 1 module V2P386 corresponds to the level 1 module A; a level 1 module V2P694 corresponds to the level 1 module B; and a level 1 module V2P319 corresponds to the level 1 module C.

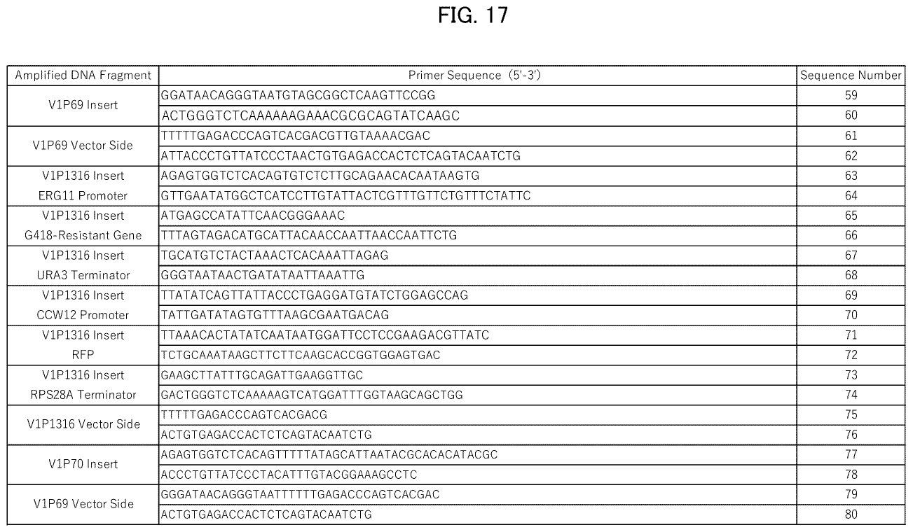

FIG. 17 is an explanatory diagram collectively showing primers used for cloning of DNA fragments required for production of the respective level 1 modules shown in FIG. 16. The respective “amplified DNA fragments” shown in FIG. 17 were amplified by PCR using these primers. A procedure of producing each of the level 1 modules first amplified a sequence of approximately 900 bp from the end of an ORF in ECM38 and a sequence of 300 bp downstream of a 3′ side of a protein coding region (CDS) in ECM 38, which were partial sequences of the CDS in ECM38 for homologous recombination with the yeast genome, by PCR using the S. cerevisiae BY4742 strain genome or the synthetic DNA as the template. The primers used are shown in FIG. 17. The procedure then sequentially bound the target DNA fragments by using the NEBuilder HiFi DNA Assembly Cloning Kit and subcloned the bound DNA fragments in a vector corresponding to the destination vector for level 0 of the Golden Gate method (shown in FIG. 13). The obtained module was called a level 0 module V1P69. This level 0 module is used for obtaining the level 1 module A (V2P386).

The procedure also subcloned a G418-Resistatnt gene (GenBank ID HQ154010.1) (G418 marker) fused with an ERG11 promoter and a URA3 terminator and an RFP gene fused with the CCW12 promoter and the RPS28A terminator, in the destination vector for level 0 (shown in FIG. 13). The obtained module was called a level 0 module V1P1316. This level 0 module is used for obtaining the level 1 module (V2P319).

The procedure also subcloned a DNA fragment including a homologous recombination region in a range of approximately 850 bp from a 301 bp on a 3′-terminal side in the CDS of the ECM38 gene in the yeast genomic DNA as the introduction object, in the destination vector for level 0 (shown in FIG. 13). The obtained module was called a level 0 module V1P70. This level 0 module is used for obtaining the level 1 module (V2P319).

The level 1 modules were produced by the Golden Gate reaction (using BsaI as the restriction enzyme) of the vectors of the respective level 0 modules and the corresponding destination vectors for level 1 shown in FIG. 16. The three different level 1 modules obtained were used in combination for introduction into the yeast genome.

### (Production of Excised Sequence-Introduced Yeast)

Three different DNA fragments for homologous recombination were amplified by using the three different level 1 modules (V2P386, V2P694, and V2P319) for production of the “excised sequence-introduced yeast”, and The Saccharomyces cerevisiae BY4742 strain was transformed with the amplified DNA fragments. The transformed strain was applied to a G418-containing YPD agar medium, and grown colonies were purified. A strain (Uz4261) having an expected homologous recombination (shown in FIG. 15) was selected as the “excised sequence-introduced yeast” by PCR. The transformation for obtaining the excised sequence-introduced yeast described above and transformation for further introducing the target gene-introducing plasmid into this excised sequence-introduced yeast as described below were performed by the method of Akada et al., (BioTechniques 28, 854, 2000).

### <Transient Expression and Site-Specific Recombination Reaction of Target Gene>

A transformed strain (Uz4267) was produced by transforming the “excised sequence-introduced yeast”, Uz4261 strain, obtained as described above with the target gene-introducing plasmid described above. The Uz4267 strain was then seeded at a concentration of forming a single colony on a YPGal (galactose as the carbon source) agar medium, and the Cre gene (the target gene 12) and the homing nuclease gene (the endonuclease gene 16) were simultaneously induced.

The above induction is expected to cause expression of the Cre gene (the target gene 12) and the resulting progress of a site-specific recombination reaction in the yeast genome and is also expected to cause expression of the homing nuclease gene (the endonuclease gene 16) and cutting of the target gene-introducing plasmid, so as to be removed. The progress of this reaction as expected loses the RFP gene in the “excised sequence 242” incorporated into the genomic DNA of the host cell and also loses the GFP gene included in the target gene-introducing plasmid. This causes colonies to become white colonies. The progress of only the removal of the target gene-introducing plasmid without the site-specific recombination reaction with the genomic DNA of the host cell, on the other hand, the host cell keeps the RFP gene. This causes colonies to become red colonies. In the case of no removal of the target gene-introducing plasmid, expression of the GFP gene causes colonies to become green colonies.

The expression types of all the colonies were checked: the ratio of the white colonies was 78.5%; the ratio of the red colonies was 21.5%; and no green colonies were observed. The plasmid retention rate is thus expected to be lower than 1%. It was confirmed that the expected reactions, i.e., the site-specific recombination and the removal of the plasmid, occurred simultaneously in a high ratio, about 80%, of the colonies.

The strains of the white colonies were checked for the G418 sensitivity and the nourseothricin sensitivity. All the strains of the white colonies showed these sensitivities. This proves the validity of the above confirmation of the expression types with regard to the site-specific recombination reaction and the removal of the plasmid.

The present disclosure is not limited to the embodiments described above but may be implemented in a variety of configurations without departing from the subject matter or the scope of the present disclosure. For example, the technical features of the embodiments corresponding to the technical features of the respective aspects described in Summary may be appropriately changed, altered, replaced or combined, in order to solve part or the entirety of the problems described above or in order to achieve part or the entirety of the advantageous effects described above. The technical features may be appropriately omitted unless the technical features are mentioned as essential in the description hereof.

### REFERENCE SIGNS LIST

10, 110, 210 . . . target gene-introducing plasmid
12 . . . target gene
14 . . . specific sequence
16 . . . endonuclease gene
18, 18A, 18B . . . inducible promoter
20 . . . host cell
32 . . . recombinase
36 . . . endonuclease
40 . . . vector for genome introduction
42 . . . genome introducing sequence
44 . . . target gene-introducing plasmid
46, 47 . . . homologous sequence
48 . . . recombination sequence
232 . . . . Cre recombinase
242 . . . excised sequence

1. A gene expression method, comprising:
providing a target gene-introducing plasmid where a target gene that is to be introduced into and to be expressed in a host cell and an endonuclease gene that specifically recognizes a specific sequence are placed to be expressible under control of one or more inducible promoters configured to induce simultaneous expression and where one or more specific sequences are introduced;
transforming the host cell with the target gene-introducing plasmid; and
culturing the transformed host cell under an inducing condition of the inducible promoter, so as to cause expression of the target gene and the endonuclease gene and to cause an endonuclease expressed by the expression of the endonuclease gene to cut the specific sequence and thereby remove the target gene-introducing plasmid.

2. The gene expression method according to claim 1,
wherein a gene for modifying a nucleic acid included in the host cell is used as the target gene.

3. The gene expression method according to claim 2,
wherein a gene of an enzyme that modifies a genomic DNA of the host cell by a homologous recombination reaction is used as the target gene.

4. The gene expression method according to claim 3,
wherein a gene coding a λ-Red recombinase system is used as the target gene.

5. The gene expression method according to claim 3,
wherein a plasmid that has two or more specific sequences and that includes a sequence, which is introduced in between the two specific sequences and which allows for homologous recombination with the genomic DNA of the host cell, is used as the target gene-introducing plasmid.

6. The gene expression method according to claim 1,
wherein a homing endonuclease gene is used as the endonuclease gene.

7. The gene expression method according to claim 1,
wherein a single inducible promoter is used as the one or more inducible promoters configured to induce simultaneous expression.

8. The gene expression method according to claim 7,
wherein a prokaryotic cell is used as the host cell, and
a plasmid where the target gene and the endonuclease gene are placed in series downstream of the single inducible promoter, is used as the target gene-introducing plasmid.

9. The gene expression method according to claim 7,
wherein a eukaryotic cell is used as the host cell,
a bidirectional promoter is used as the single inducible promoter, and
a plasmid where the target gene and the endonuclease gene are placed across the bidirectional promoter to be located on respective sides of the bidirectional promoter and are connected such as to be simultaneously expressible by induction of the bidirectional promoter, is used as the target gene-introducing plasmid.

10. The gene expression method according to claim 7,
wherein a eukaryotic cell is used as the host cell, and
a plasmid where the target gene and the endonuclease gene are placed in series downstream of the single inducible promoter and are connected with each other by a linker such as to be controlled by the single inducible promoter and to be transcribed simultaneously, is used as the target gene-introducing plasmid.

11. The gene expression method according to claim 4,
wherein a plasmid that has two or more specific sequences and that includes a sequence, which is introduced in between the two specific sequences and which allows for homologous recombination with the genomic DNA of the host cell, is used as the target gene-introducing plasmid.

12. The gene expression method according to claim 2,
wherein a single inducible promoter is used as the one or more inducible promoters configured to induce simultaneous expression.

13. The gene expression method according to claim 12,
wherein a prokaryotic cell is used as the host cell, and
a plasmid where the target gene and the endonuclease gene are placed in series downstream of the single inducible promoter, is used as the target gene-introducing plasmid.

14. The gene expression method according to claim 12,
wherein a eukaryotic cell is used as the host cell,
a bidirectional promoter is used as the single inducible promoter, and
a plasmid where the target gene and the endonuclease gene are placed across the bidirectional promoter to be located on respective sides of the bidirectional promoter and are connected such as to be simultaneously expressible by induction of the bidirectional promoter, is used as the target gene-introducing plasmid.

15. The gene expression method according to claim 12,
wherein a eukaryotic cell is used as the host cell, and
a plasmid where the target gene and the endonuclease gene are placed in series downstream of the single inducible promoter and are connected with each other by a linker such as to be controlled by the single inducible promoter and to be transcribed simultaneously, is used as the target gene-introducing plasmid.


SEQUENCE LISTING (80 sequences)

SEQ ID NO: 1 - DNA, 60 bp, synthetic construct
  source 1..60: mol_type = other DNA
gtagtgactcgcggccagttagggataacagggtaatttccaacttccgccccggcacag

SEQ ID NO: 2 - DNA, 41 bp, synthetic construct
  source 1..41: mol_type = other DNA
tgtcctggctggactgaaaattatagagtcgcaacggcctg

SEQ ID NO: 3 - DNA, 20 bp, synthetic construct
  source 1..20: mol_type = other DNA
ccagccaggacagaaatgcc

SEQ ID NO: 4 - DNA, 66 bp, synthetic construct
  source 1..66: mol_type = other DNA
agaaaaaacgcccggtgtgcaagaccgagcgttctgaacaattatttgccgactaccttggtgatc

SEQ ID NO: 5 - DNA, 109 bp, synthetic construct
  source 1..109: mol_type = other DNA
ccgggcgttttttctttgtgagtccatgcacggcgctaggtgtgatatcgtacacttgggagaagtcagatacgattgcggctcagtatgacgatttttgataattatg

SEQ ID NO: 6 - DNA, 24 bp, synthetic construct
  source 1..24: mol_type = other DNA
aattgctggctgccaatgacaaac

SEQ ID NO: 7 - DNA, 60 bp, synthetic construct
  source 1..60: mol_type = other DNA
tcattggcagccagcaattaccctgttatccctataatgtgagttagctcactcattagg

SEQ ID NO: 8 - DNA, 44 bp, synthetic construct
  source 1..44: mol_type = other DNA
ctagtatttctcctctttctctagtatgtgtgaaattgttatcc

SEQ ID NO: 9 - DNA, 36 bp, synthetic construct
  source 1..36: mol_type = other DNA
aaagaggagaaatactagatggattcctccgaagac

SEQ ID NO: 10 - DNA, 43 bp, synthetic construct
  source 1..43: mol_type = other DNA
tggatctatcaacaggagtctcaagcaccggtggagtgacgac

SEQ ID NO: 11 - DNA, 28 bp, synthetic construct
  source 1..28: mol_type = other DNA
gactcctgttgatagatccagtaatgac

SEQ ID NO: 12 - DNA, 37 bp, synthetic construct
  source 1..37: mol_type = other DNA
gagctagccgtcaacattctcaccaataaaaaacgcc

SEQ ID NO: 13 - DNA, 73 bp, synthetic construct
  source 1..73: mol_type = other DNA
gttgacggctagctcagtcctaggtattgtgctagcaaagaggagaaaatgtcacatctcgcagaactggttg

SEQ ID NO: 14 - DNA, 60 bp, synthetic construct
  source 1..60: mol_type = other DNA
aaaaccccgccgaagcggggttttcggcgttatttaaactgtttgaggaaacgcagatcg

SEQ ID NO: 15 - DNA, 49 bp, synthetic construct
  source 1..49: mol_type = other DNA
gcttcggcggggttttgccgcctgtcagaccaagtttactcatatatac

SEQ ID NO: 16 - DNA, 24 bp, synthetic construct
  source 1..24: mol_type = other DNA
taacgcaggaaagaacatgtgagc

SEQ ID NO: 17 - DNA, 49 bp, synthetic construct
  source 1..49: mol_type = other DNA
gttctttcctgcgttattaagacccactttcacatttaagttgtttttc

SEQ ID NO: 18 - DNA, 35 bp, synthetic construct
  source 1..35: mol_type = other DNA
atgtatatctccttcttaaaagatcttttgaattc

SEQ ID NO: 19 - DNA, 49 bp, synthetic construct
  source 1..49: mol_type = other DNA
gaaggagatatacataatgaaaaatattaaaaagaaccaggtgatgaac

SEQ ID NO: 20 - DNA, 45 bp, synthetic construct
  source 1..45: mol_type = other DNA
cagtattaatatccatctactttaaaaaggtttcgctactgattg

SEQ ID NO: 21 - DNA, 31 bp, synthetic construct
  source 1..31: mol_type = other DNA
atggatattaatactgaaactgagatcaagc

SEQ ID NO: 22 - DNA, 79 bp, synthetic construct
  source 1..79: mol_type = other DNA
ggtcagtgacctcttaagagcgaaaaaaccccgccgaagcggggttttttgcgttaactcaacagaagatgctttgtgc

SEQ ID NO: 23 - DNA, 76 bp, synthetic construct
  source 1..76: mol_type = other DNA
aagaggtcactgacctaacaaaaaaaaaaccccgcccctgacagggcggggttttttttggaggtactggcctagc

SEQ ID NO: 24 - DNA, 67 bp, synthetic construct
  source 1..67: mol_type = other DNA
ggccgcgagtcactactaaactgtctctcccgggccacgacgctaggccagtacctccaaaaaaaac

SEQ ID NO: 25 - DNA, 41 bp, synthetic construct
  source 1..41: mol_type = other DNA
catgttctttcctgcgttaagcttgcaaattaaagccttcg

SEQ ID NO: 26 - DNA, 26 bp, synthetic construct
  source 1..26: mol_type = other DNA
ttagttatgtcacgcttacattcacg

SEQ ID NO: 27 - DNA, 38 bp, synthetic construct
  source 1..38: mol_type = other DNA
gcgtgacataactaatcaatcaccatcttccaacaatc

SEQ ID NO: 28 - DNA, 25 bp, synthetic construct
  source 1..25: mol_type = other DNA
gctttcgaaagaactgatttcgatc

SEQ ID NO: 29 - DNA, 66 bp, synthetic construct
  source 1..66: mol_type = other DNA
agttctttcgaaagcctgcaaaacttgtgcttgtacacctcgaatgttagtaaatggatatctttt

SEQ ID NO: 30 - DNA, 67 bp, synthetic construct
  source 1..67: mol_type = other DNA
gctaagcaggctttggcatgtataacaaacactgatttttgttttgagttttaaaagatatccattt

SEQ ID NO: 31 - DNA, 23 bp, synthetic construct
  source 1..23: mol_type = other DNA
caaagcctgcttagctctttcac

SEQ ID NO: 32 - DNA, 30 bp, synthetic construct
  source 1..30: mol_type = other DNA
atgtctaacttgttgactgttcatcagaac

SEQ ID NO: 33 - DNA, 59 bp, synthetic construct
  source 1..59: mol_type = other DNA
agtcaacaagttagacatttatattgaattttcaaaaattcttactttttttttggatg

SEQ ID NO: 34 - DNA, 35 bp, synthetic construct
  source 1..35: mol_type = other DNA
tatagttttttctccttgacgttaaagtatagagg

SEQ ID NO: 35 - DNA, 50 bp, synthetic construct
  source 1..50: mol_type = other DNA
aggagaaaaaactatagtatgtatctataaatttgaaaccaatattaggc

SEQ ID NO: 36 - DNA, 59 bp, synthetic construct
  source 1..59: mol_type = other DNA
cttcttctttggcatgatttattaaatgttctatgataaagtaaaaagtaaaaacatac

SEQ ID NO: 37 - DNA, 59 bp, synthetic construct
  source 1..59: mol_type = other DNA
atgccaaagaagaagagaaaggtaaaaaacattaagaaaaaccaagttatgaatttggg

SEQ ID NO: 38 - DNA, 25 bp, synthetic construct
  source 1..25: mol_type = other DNA
tcatttcaagaaggtttcggaggag

SEQ ID NO: 39 - DNA, 36 bp, synthetic construct
  source 1..36: mol_type = other DNA
aaccttcttgaaatgatcatctttgaaggcttgtgc

SEQ ID NO: 40 - DNA, 24 bp, synthetic construct
  source 1..24: mol_type = other DNA
taacgcaggaaagaacatgtgagc

SEQ ID NO: 41 - DNA, 35 bp, synthetic construct
  source 1..35: mol_type = other DNA
agtaaatacataagcgtgaggatgtatctggagcc

SEQ ID NO: 42 - DNA, 29 bp, synthetic construct
  source 1..29: mol_type = other DNA
tattgatatagtgtttaagcgaatgacag

SEQ ID NO: 43 - DNA, 42 bp, synthetic construct
  source 1..42: mol_type = other DNA
aacactatatcaataatgagtaagggcgcagaattgttcaac

SEQ ID NO: 44 - DNA, 36 bp, synthetic construct
  source 1..36: mol_type = other DNA
tgcaaataagcttctcttgtacaactcgtccattcc

SEQ ID NO: 45 - DNA, 27 bp, synthetic construct
  source 1..27: mol_type = other DNA
gaagcttatttgcagattgaaggttgc

SEQ ID NO: 46 - DNA, 23 bp, synthetic construct
  source 1..23: mol_type = other DNA
gtcatggatttggtaagcagctg

SEQ ID NO: 47 - DNA, 79 bp, synthetic construct
  source 1..79: mol_type = other DNA
taccaaatccatgactagggataacagggtaatacgtacgtattaccctgttatccctaggtccttttcatcacgtgct

SEQ ID NO: 48 - DNA, 38 bp, synthetic construct
  source 1..38: mol_type = other DNA
tgaaactatgattcctggacggatcgcttgcctgtaac

SEQ ID NO: 49 - DNA, 30 bp, synthetic construct
  source 1..30: mol_type = other DNA
aggaatcatagtttcatgattttctgttac

SEQ ID NO: 50 - DNA, 45 bp, synthetic construct
  source 1..45: mol_type = other DNA
caggatgacgcctaaaaagattctctttttttatgatatttgtac

SEQ ID NO: 51 - DNA, 20 bp, synthetic construct
  source 1..20: mol_type = other DNA
ttaggcgtcatcctgtgctc

SEQ ID NO: 52 - DNA, 35 bp, synthetic construct
  source 1..35: mol_type = other DNA
cacactaaattaataatgaagatttcggtgatccc

SEQ ID NO: 53 - DNA, 33 bp, synthetic construct
  source 1..33: mol_type = other DNA
tattaatttagtgtgtgtatttgtgtttgtgtg

SEQ ID NO: 54 - DNA, 40 bp, synthetic construct
  source 1..40: mol_type = other DNA
acaaataggggttccgcgacgacatcgtcgaatatgattc

SEQ ID NO: 55 - DNA, 27 bp, synthetic construct
  source 1..27: mol_type = other DNA
cgcggaacccctatttgtttatttttc

SEQ ID NO: 56 - DNA, 37 bp, synthetic construct
  source 1..37: mol_type = other DNA
aacttggtctgacagttaccaatgcttaatcagtgag

SEQ ID NO: 57 - DNA, 37 bp, synthetic construct
  source 1..37: mol_type = other DNA
ctgtcagaccaagtttactcatatatactttagattg

SEQ ID NO: 58 - DNA, 29 bp, synthetic construct
  source 1..29: mol_type = other DNA
taacgcaggaaagaacatgtgagcaaaag

SEQ ID NO: 59 - DNA, 34 bp, synthetic construct
  source 1..34: mol_type = other DNA
ggataacagggtaatgtagcggctcaagttccgg

SEQ ID NO: 60 - DNA, 35 bp, synthetic construct
  source 1..35: mol_type = other DNA
actgggtctcaaaaaagaaacgcgcagtatcaagc

SEQ ID NO: 61 - DNA, 34 bp, synthetic construct
  source 1..34: mol_type = other DNA
tttttgagacccagtcacgacgttgtaaaacgac

SEQ ID NO: 62 - DNA, 46 bp, synthetic construct
  source 1..46: mol_type = other DNA
attaccctgttatccctaactgtgagaccactctcagtacaatctg

SEQ ID NO: 63 - DNA, 40 bp, synthetic construct
  source 1..40: mol_type = other DNA
agagtggtctcacagtgtctcttgcagaacacaataagtg

SEQ ID NO: 64 - DNA, 48 bp, synthetic construct
  source 1..48: mol_type = other DNA
gttgaatatggctcatccttgtattactcgtttgttctgtttctattc

SEQ ID NO: 65 - DNA, 23 bp, synthetic construct
  source 1..23: mol_type = other DNA
atgagccatattcaacgggaaac

SEQ ID NO: 66 - DNA, 38 bp, synthetic construct
  source 1..38: mol_type = other DNA
tttagtagacatgcattacaaccaattaaccaattctg

SEQ ID NO: 67 - DNA, 29 bp, synthetic construct
  source 1..29: mol_type = other DNA
tgcatgtctactaaactcacaaattagag

SEQ ID NO: 68 - DNA, 26 bp, synthetic construct
  source 1..26: mol_type = other DNA
gggtaataactgatataattaaattg

SEQ ID NO: 69 - DNA, 39 bp, synthetic construct
  source 1..39: mol_type = other DNA
ttatatcagttattaccctgaggatgtatctggagccag

SEQ ID NO: 70 - DNA, 29 bp, synthetic construct
  source 1..29: mol_type = other DNA
tattgatatagtgtttaagcgaatgacag

SEQ ID NO: 71 - DNA, 42 bp, synthetic construct
  source 1..42: mol_type = other DNA
ttaaacactatatcaataatggattcctccgaagacgttatc

SEQ ID NO: 72 - DNA, 37 bp, synthetic construct
  source 1..37: mol_type = other DNA
tctgcaaataagcttcttcaagcaccggtggagtgac

SEQ ID NO: 73 - DNA, 27 bp, synthetic construct
  source 1..27: mol_type = other DNA
gaagcttatttgcagattgaaggttgc

SEQ ID NO: 74 - DNA, 40 bp, synthetic construct
  source 1..40: mol_type = other DNA
gactgggtctcaaaaagtcatggatttggtaagcagctgg

SEQ ID NO: 75 - DNA, 22 bp, synthetic construct
  source 1..22: mol_type = other DNA
tttttgagacccagtcacgacg

SEQ ID NO: 76 - DNA, 28 bp, synthetic construct
  source 1..28: mol_type = other DNA
actgtgagaccactctcagtacaatctg

SEQ ID NO: 77 - DNA, 45 bp, synthetic construct
  source 1..45: mol_type = other DNA
agagtggtctcacagtttttatagcattaatacgcacacatacgc

SEQ ID NO: 78 - DNA, 34 bp, synthetic construct
  source 1..34: mol_type = other DNA
accctgttatccctacatttgtacggaaagcctc

SEQ ID NO: 79 - DNA, 37 bp, synthetic construct
  source 1..37: mol_type = other DNA
gggataacagggtaattttttgagacccagtcacgac

SEQ ID NO: 80 - DNA, 28 bp, synthetic construct
  source 1..28: mol_type = other DNA
actgtgagaccactctcagtacaatctg